SEQUENCE LISTING (16 sequences)

SEQ ID NO: 1 - DNA, 26 bp, Artificial
  PCR primer
ccggatccatgtgacagacagtgtta

SEQ ID NO: 2 - DNA, 27 bp, Artificial
  PCR primer
aagcccgggacttgtactaactgtttc

SEQ ID NO: 3 - DNA, 27 bp, Artificial
  PCR primer
tatcggatccaaatcataagaaaggag

SEQ ID NO: 4 - DNA, 26 bp, Artificial
  PCR primer
gaagatctatattgttcatcccattg

SEQ ID NO: 5 - DNA, 26 bp, Artificial
  PCR primer
gcacccgggaggagatatgcagtttg

SEQ ID NO: 6 - DNA, 22 bp, Artificial
  PCR primer
gactgcagcacaaatggtcaag

SEQ ID NO: 7 - DNA, 27 bp, Artificial
  PCR primer
tatcggatccaaatcataagaaaggag

SEQ ID NO: 8 - DNA, 26 bp, Artificial
  PCR primer
gaagatctatattgttcatcccattg

SEQ ID NO: 9 - DNA, 22 bp, Artificial
  PCR primer
tagacattgtaggttggttttg

SEQ ID NO: 10 - DNA, 24 bp, Artificial
  PCR primer
gcacaggttatcaacacgtttgac

SEQ ID NO: 11 - DNA, 22 bp, Artificial
  PCR primer
atccggccatttctgaagagaa

SEQ ID NO: 12 - DNA, 22 bp, Artificial
  PCR primer
tctagttacttcataagttttg

SEQ ID NO: 13 - DNA, 24 bp, Artificial
  PCR primer
ttgcgggtttgtgtttccatattg

SEQ ID NO: 14 - DNA, 22 bp, Artificial
  PCR primer
attgataaggggttattagggg

SEQ ID NO: 15 - DNA, 21 bp, Artificial
  PCR primer
ggtttcgctcatgtgttgagc

SEQ ID NO: 16 - DNA, 25 bp, Artificial
  PCR primer
cattgcggacgtttttaatgtactg

(19) United States
(12) Patent Application Publication
(10) Pub. No.: US 2005/0120416 A1
(43) Pub. Date: Jun. 2, 2005

(54) Novel method for the production of hybrid maize seeds

(76) Inventors: Pascual Perez, Chanonat (FR); Denise Gerentes, Le Crest (FR); Sylvain Levadoux, Chatel-Guyon (FR)

Correspondence Address: CONNOLLY BOVE LODGE & HUTZ, LLP, P O BOX 2207, WILMINGTON, DE, 19899, US

(21) Appl. No.: 10/506,946

(22) PCT Filed: Mar. 5, 2003
(86) PCT No.: PCT/FR03/00711

(30) Foreign Application Priority Data
Mar. 8, 2002 (FR) 02/02968

(51) Int. Cl. A01H 1/00; C12N 15/82; A01H 5/00

(52) U.S. Cl. 800/287; 800/320.1

(57) ABSTRACT

The invention relates to a method for production and multiplication of maize plants homozygous for a transgene which confers male sterility, useful in the production of hybrid maize seed.

The present invention relates to a method for the production and multiplication of young maize plants homozygous for a transgene conferring male sterility, that are useful for the production of hybrid maize seeds.

The production of hybrids via “sexual hybridization” of parents having parents having different genetic backgrounds is of great importance in modern agricultural practice. In fact, crossing between plants that are of the same species but not related produces a lineage which manifests characteristics, such as yield or disease resistance, greater than those of the parents: this is the heterosis or hybrid vigor effect. Thus, the production of hybrids is often used to improve both the quality and the yield of crop plants.

In addition, these descendants are genetically uniform, in particular in terms of the characteristics of productivity, sensitivity to torrential rain and to drought, initial vigor and sensitivity to diseases and to pests, and are therefore advantageous for farmers.

Without human intervention, the production of hybrids is limited by phenomena of self-fertilization. The production of hybrid seeds therefore makes it necessary to favor cross-fertilization by preventing self-pollination by mechanical, chemical or genetic techniques. The various approaches for preventing self-fertilization by controlling pollination include in particular:
manual or mechanical “castration” or chemical inactivation of the male organs of the plant,
the use of a modified plant exhibiting recessive nuclear male sterility,
the use of a modified plant exhibiting recessive cytoplasmic male sterility,
the use of a dominant nuclear male sterile plant (artificial male sterility or AMS).

However, some of these techniques are not completely effective and sometimes even difficult to implement.
“Castration” is expensive and can cause losses in yield in the event of error.

The effectiveness of using chemical agents, which is less expensive, depends on the environmental conditions at the time of application of the gametocide, which leads the seed producer to take considerable risks each season.

The use of a recessive nuclear male sterile plant is not easy to implement, in particular due to the maintaining of the male sterile characteristics, which requires reselection of male sterile plants in the descendents obtained by self-fertilization of a plant heterozygous for the recessive male sterility gene. To be effective, this system therefore involves the use of a marker that is closely linked to the male sterility gene and readily identifiable.

Male sterility can be “acquired”, i.e. it is independent of any genetic manipulation via the recombinant DNA process. Cytoplasmic male sterility can be distinguished from nuclear male sterility.

“Cytoplasmic” male sterility (“CMS”) is related to changes in the organization and the expression of the mitochondrial genome, nuclear male sterility results from mutations in the genome of the cell's nucleus. However, CMS is not complete, 2/1000 of the plants remaining fertile. The loss of cytoplasmic genetic diversity when selectors use the same cytoplasm in their selection program can, moreover, be handicapped.

Male sterility can be “artificial” (“AMS”), i.e. it is induced by the expression of a gene conferring male sterility (AMS gene) which is inserted either into the mitochondrial genome (cytoplasmic male sterility) or into the nuclear genome (nuclear male sterility).

The AMS system makes it possible to avoid the problems associated with the other methods. As a result, unlike CMS, the AMS system does not depend on the existence of a mutant. Maintenance of the sterile characteristic of the male line can be obtained using a dominant male sterility gene linked to a marker gene which allows the selection of the artificial male sterile plants.

The problem of maintaining the male sterile characteristic in a plant line has been partly solved in patent application WO 95/34634, which envisions crossing between a plant heterozygous for a dominant male sterility gene and a plant which restores fertility, and then selecting the male sterile seeds. In that application, the use of a marker, linked to the fertility-restoring gene, which influences the regulation of the genes encoding the regulatory enzymes for anthocyanin synthesis, makes it possible to differentiate, by means of the color, the male sterile seeds from the male fertile seeds.

However, the separating system described in that patent application does not make it possible to reliably separate hybrid seeds on a large scale.

In addition, the general application of this technology is limited insofar as the choice of the coloration marker is conditioned by the genotype of the plant containing the fertility-restoring gene. In particular, only plants whose genotype does not condition a visible production of anthocyanin in the seeds can be used.

The present invention therefore proposes a method for the production and multiplication of young maize plants homozygous for a transgene conferring artificial nuclear male sterility, allowing hybrid seeds to be produced, and readily applicable on a large scale. An advantage of the method proposed is that its application is not conditioned by the genotype of the plants used.

This approach is based in particular on combining the fertility characteristic with a “small seed” phenotype. The “small seed” phenotype is preferentially obtained by the expression of the shrunken 2 and brittle 2 genes in antisense orientation. The brittle 2 gene encodes one of the 2 subunits of ADP glucose pyrophosphorylase, an enzyme involved in starch synthesis. The fragment was obtained by the RT-PCR technique from a maize ear total RNA extract and according to the Genbank data (accession No. S72425). This fragment represents an incomplete cDNA of the brittle 2 gene. The shrunken 2 gene encodes the other subunit of ADP glucose pyrophosphorylase. The fragment was obtained by the RT-PCR technique from a maize ear total RNA extract and according to the Genbank data (accession No. S72425). This fragment contains the complete sequence of the coding region of the shrunken 2 gene.

The inhibition of these two genes for wrinkled mutants of reduced size and weight makes it possible to obtain maize seeds of reduced density and/or size. Such seeds can be readily separated by sieving and/or densimetric separation, for example using a densimetric table or column, or by flotation, which makes it possible to use a reliable, simple selection that is automated and therefore applicable on a large scale.

In the context of the present invention, the expression “heterozygous for the AMS transgene” denotes a young maize plant made male sterile by incorporation into its genome of a single copy of a transgene conferring artificial nuclear male sterility (“AMS”). Unless otherwise indicated, this young plant does not contain the fertility-restoring gene linked to a “small seed” phenotype marker.

In the context of the present invention, the expression “homozygous for the AMS transgene” denotes a young maize plant made male sterile by incorporation into its genome two copies, located at the same place on each of the sister chromosomes, of a transgene conferring artifical nuclear male sterility (AMS). Unless otherwise indicated, this young plant does not contain the fertility-restoring gene linked to a “small seed” phenotype marker.

The expression “fertility-restoring young maize plant comprising in its genome a fertility-restoring gene linked to a “small seed” phenotype marker” is intended to mean a young maize plant heterozygous or homozygous for said fertility-restoring gene linked to a “small seed” phenotype marker. Preferably, said young maize plant is heterozygous. Unless otherwise indicated, this young plant does not contain a transgene conferring artificial nuclear male sterility (AMS).

The expression “young maize plant comprising in its genome an AMS transgene” is intended to mean a young maize plant heterozygous or homozygous for said AMS transgene.

The expression “young maize plant having a wild-type genotype” is intended to mean a young maize plant which contains in its genome neither a transgene conferring artificial nuclear male sterility (AMS) nor a fertility-restoring gene linked to a “small seed” phenotype marker. Preferably, said young maize plant having a wild-type genotype belongs to an elite line.

The expression “small seed” phenotype is intended to mean seeds for which the density and/or the size and/or the mass is (are) smaller than that of a seed of normal size, preferably from 40 to 50% smaller. In the context of the present invention, these seeds may be referred to as “deficient seeds” or “wrinkled seeds”.

The expression “small seed phenotype marker” or “gene which confers a small seed phenotype” or “gene encoding a small seed phenotype” is intended to mean any gene, in the sense orientation or in the antisense orientation, which, when it is expressed in the plant, confers a “small seed” phenotype. Among these markers mention may in particular be made of the shrunken 2, brittle 2 and shrunken 1 genes and the miniature locus 1 which encodes an invertase; and, more generally, any gene which makes it possible to decrease the starch content and does not impair the seed viability.

The expression “seed of normal size” or “normal seed” or “seed of normal phenotype” is intended in particular to mean a seed whose size and/or density and/or mass has or have not been modified, in particular by integration into the genome of the plant of a “small seed” phenotype marker.

The term “elite line” is intended to mean a line having a substantial agronomic and commercial potential, at a given period.

The term “linkage” or “genetic linkage” is intended to mean a genetic distance sufficiently small for the frequencies of recombination during meiosis to be negligible.

The term “protein of interest” is intended to mean a protein of human or animal origin which may be of therapeutic and/or prophylactic interest, such as collagen, gastric lipase, antibodies, etc.

One of the aims of the invention is therefore to propose a method for the production of hybrid maize seeds by crossing a young maize plant heterozygous for a transgene conferring artificial nuclear male sterility (“AMS”) with a young maize plant having a wild-type genotype. The system according to the present invention, which advantageously makes it possible to maintain the male sterile characteristic of young maize plants, is applicable independently of the genotype of the young maize plants used.

The solution provided by the invention consists in initially producing maize seeds homozygous for a transgene conferring artificial nuclear male sterility (“AMS”) and heterozygous for a fertility-restoring gene linked to a “small seed” phenotype marker. Using these seeds, selectors can easily introgress the genotype homozygous for the AMS transgene and heterozygous for a fertility-restoring gene linked to a “small seed” phenotype marker into a maize line having a wild-type genotype, and in particular into an elite line of interest, by successive backcrosses. A converted elite line homozygous for a transgene conferring artificial nuclear male sterility (“AMS”) and heterozygous for a fertility-restoring gene linked to a “small seed” phenotype marker can thus be obtained. Self-fertilization of young maize plants derived from these seeds then makes it possible to produce:
seeds homozygous for the AMS transgene generating male sterile young maize plants which, when crossed with young maize plants having a wild-type genotype, and in particular young plants belonging to an elite line used for conversion, where appropriate, will produce seeds heterozygous for the AMS transgene. Fertilization of male sterile young maize plants derived from these seeds, by an unrelated young maize plant having a wild-type genotype then produces hybrid seeds which benefit from the vigor effect (heterosis),
seeds homozygous for the AMS transgene and heterozygous or homozygous for the fertility-restoring gene linked to a “small seed” phenotype marker, generating non-male sterile young maize plants, the self-fertilization of which produces in particular seeds homozygous for the AMS transgene and heterozygous for the fertility-restoring gene linked to a “small seed” phenotype marker, thus ensuring renewal of the male sterile young maize plants of interest.

Advantageously, when the AMS transgene is genetically linked to a gene encoding a protein of interest, the hybrid seeds homozygous for the AMS transgene are found to be particularly useful for generating young maize plants producing said protein of interest.

In the context of the present invention, selection of the maize seeds by means of the “small seed” phenotype can in particular be carried out by a method of size separation or densimetric separation.

A size separation can be carried out so as to separate the seeds as a function of their length, using for example an indent separator, of their width, with for example a disk separator, or of their thickness, using for example a calibrator.

The principle of densimetric separation is based on separation of the seeds according to their density and uses in particular a densimetric column or a densimetric table, or a flotation system.

More particularly,
the indent separator is made up of a rotating horizontal cylinder. The separation is carried out by centrifugal force. The smallest seeds enter into the indents (which cover the inside of the cylinder) and are kept there by the centrifugal force;
the disk separator is made up of thick disks arranged vertically with indents of appropriate size hollowed out in their thickness;
the most conventional calibrators are thickness-calibrators made up of flat or cylindrical sieves with elongated perforations which force the seed to present itself according to its smallest thickness in order to pass through. The round-perforation calibrator works on the same principle, but the round orifices with small “notches” inside the cylinder forcing the seed to present itself vertically in order to pass through the sieve;
the densimetric column comprises a vibrating distributor which introduces the mixture of seeds to be separated halfway up a hollow column in which there is an ascending homogeneous stream of air. The heavy particles fall whereas the lighter particles rise. The separation is thus effected;
a densimetric table makes it possible to separate bodies of the same size but with a different specific weight. The specific weight corresponds to a measurement of the mass of a quantity of seeds relative to its volume. The principle of the device is a workstation (often consisting of a lattice made of metal wire or textile) through which a uniform air stream passes, which fluidifies the mixture of seeds and causes the stratification thereof schematically into two layers. The heavy products remain close to the table and the light products above. Separation of the two layers is obtained by adjusting the incline of the workstation (in two directions) and by a transverse agitating movement backward and forward. A densimetric table such as that sold by the company Cimbria Heid GmbH (Stockerau, Austria) may, for example, be used;
the flotation system is a system which makes it possible to separate the seeds as a function of their density and/or mass, based on their ability to float on a liquid, in particular water. The heaviest seeds (having the least ability to float) are found at the bottom and the lightest seeds (having the greatest ability to float) are found at the surface of the liquid, which enables the separation. When this system is used, the seeds should remain in contact with the water for as little time as possible, so as not to impair their germination capacity. A drying step may sometimes be necessary.

The present invention therefore proposes a method for the production of maize seeds homozygous for a transgene conferring artificial nuclear male sterility (“AMS”) and heterozygous for a fertility-restoring gene linked to a “small seed” phenotype marker, comprising the steps consisting in:
a) crossing a male sterile young maize plant heterozygous for the AMS transgene with a fertility-restoring young maize plant comprising in its genome a fertility-restoring gene linked to a “small seed” phenotype marker,
b) selecting, by means of the “small seed” phenotype, the maize seeds comprising in their genome a fertility-restoring gene linked to a “small seed” phenotype marker,
c) self-fertilizing the young maize plants derived from seeds selected according to step b),
d) selecting the seeds homozygous for the AMS transgene and heterozygous for the fertility-restoring gene linked to a “small seed” phenotype marker.

Preferably, at least one selection step in the method comprises densimetric separation, in particular using a densimetric table or column, or a flotation system.

The selection step thus carried out has the advantage of allowing a separation of the seeds, as a function of their “small seed” or “normal” phenotype, which can be readily implemented and automated.

According to a particular embodiment, step b) of the method described above may advantageously be replaced with a genotyping step. The present invention therefore also proposes a method for the production of maize seeds homozygous for a transgene conferring artificial nuclear male sterility (“AMS”) and heterozygous for a fertility-restoring gene linked to a “small seed” phenotype marker, comprising the steps consisting in:
a) crossing a male sterile young maize plant heterozygous for the AMS transgene with a fertility-restoring young maize plant comprising in its genome a fertility-restoring gene linked to a “small seed” phenotype marker,
b) genotyping the seeds obtained by means of the cross according to step a), and selecting the maize seeds comprising in their genome a fertility-restoring gene linked to a “small seed” phenotype marker,
c) self-fertilizing the young maize plants derived from the seeds genotyped according to step b),
d) selecting the seeds homozygous for the AMS transgene and heterozygous for the fertility-restoring gene linked to a “small seed” phenotype marker.

According to a particular embodiment, said young maize plant heterozygous for a transgene conferring artificial nuclear male sterility also contains a gene encoding a protein of interest, preferably genetically linked to the AMS transgene.

The implementation of the methods described above makes it possible to produce a maize seed homozygous for an AMS transgene and heterozygous for a fertility-restoring gene linked to a “small seed” phenotype marker. The present invention is therefore directed toward a maize seed homozygous for an AMS transgene and heterozygous for a fertility-restoring gene linked to a “small seed” phenotype marker, which can be obtained by one of the above methods. Preferably, the genotype that is AMS-homozygous and heterozygous for a fertility-restoring gene linked to a “small seed” phenotype marker can be introgressed into a maize line having a wild-type genotype, and in particular into an elite line of interest, by successive backcrosses.

The cultivating, and then the self-fertilizing of the young maize plants obtained from seeds above (homozygous for an AMS transgene and heterozygous for a fertility-restoring gene linked to a “small seed” phenotype marker) produces maize seeds homozygous for the AMS transgene and also maize genes homozygous for the AMS transgene and heterozygous or homozygous for the fertility-restoring gene linked to a “small seed” phenotype marker. The maize seeds homozygous for the AMS transgene can be readily differentiated from the other seeds thus produced since only they have seeds of normal phenotype.

The present invention therefore proposes a method for the production of maize seeds homozygous for a transgene conferring artificial nuclear male sterility (“AMS”), comprising the steps consisting in:
a) self-fertilizing the young maize plants derived from seeds homozygous for the AMS transgene and heterozygous for a fertility-restoring gene linked to a “small seed” phenotype marker, which can be obtained by one of the methods described above,
b) selecting seeds homozygous for the AMS transgene.

Preferably, the selection step comprises densimetric separation, in particular using a densimetric table or column, or a flotation system. This step advantageously makes it possible to separate the seeds homozygous for the AMS transgene by means of their normal seed phenotype.

According to another embodiment, the method for the production of maize seeds homozygous for a transgene conferring artificial nuclear male sterility (“AMS”), comprises the steps consisting in:
a) crossing a male sterile young maize plant heterozygous for the AMS transgene with a fertility-restoring young maize plant comprising in its genome a fertility-restoring gene linked to a “small seed” phenotype marker,
b) selecting, by means of the “small seed” phenotype, the maize seeds comprising in their genome a fertility-restoring gene linked to a “small seed” phenotype marker,
c) self-fertilizing the young maize plants derived from the seeds selected according to step b),
d) selecting seeds homozygous for the AMS transgene and heterozygous for the fertility-restoring gene linked to a “small seed” phenotype marker,
e) self-fertilizing young maize plants derived from seeds according to step d),
f) selecting seeds homozygous for the AMS transgene.

According to a particular embodiment, said young maize plant heterozygous for a transgene conferring artificial nuclear male sterility also contains a gene encoding a protein of interest, preferably genetically linked to the AMS transgene.

Step e) of this method may optionally be preceded by a step of successive backcrosses with a young maize plant having a wild-type genotype, and in particular a young maize plant belonging to an elite line, so as to convert this young maize having a wild-type genotype with the genotype homozygous for the AMS transgene and heterozygous for the fertility-restoring gene linked to a “small seed” phenotype marker. The seeds derived from this backcrossing step are then used for the continuation of the method.

Preferably, at least one selection step, and in particular step f), comprises densimetric separation, in particular using a densimetric table or column, or a flotation system. This step advantageously makes it possible to separate the seeds homozygous for the AMS transgene by means of their normal seed phenotype.

According to another variant, step b) of the method above is replaced with a genotyping step. The present invention therefore also relates to a method for the production of maize seeds homozygous for a transgene conferring artificial nuclear male sterility (“AMS”), comprising the steps consisting in:
a) crossing a male sterile young maize plant heterozygous for the AMS transgene with a fertility-restoring young maize plant comprising in its genome a fertility-restoring gene linked to a “small seed” phenotype marker,
b) genotyping the seeds obtained by means of the cross according to step a), and selecting the maize seeds comprising in their genome a fertility-restoring gene linked to a “small seed” marker,
c) self-fertilizing the young maize plants derived from the seeds genotyped according to step b),
d) selecting the seeds homozygous for the AMS transgene and heterozygous for the fertility-restoring gene linked to a “small seed” phenotype marker,
e) self-fertilizing young maize plants derived from seeds according to step d),
f) selecting seeds homozygous for the AMS transgene.

According to a particular embodiment, said young maize plant heterozygous for a transgene conferring artificial nuclear male sterility also contains a gene encoding a protein of interest, preferably genetically linked to the AMS transgene.

Step e) of this method may optionally be preceded by a step of successive backcrosses with a young maize plant having a wild-type genotype, and in particular a young maize plant belonging to an elite line, so as to convert this young maize plant having a wild-type genotype with the genotype homozygous for the AMS transgene and heterozygous for the fertility-restoring gene linked to a “small seed” phenotype marker. The seeds derived from this backcrossing step are then used for the continuation of the method.

Preferably, at least one selection step and in particular step f), comprises densimetric separation, in particular using a densimetic table or column, or a flotation system. This step advantageously makes it possible to separate the seeds homozygous for the AMS transgene by means of their normal seed phenotype.

The cultivating of seeds homozygous for the AMS transgene then makes it possible to generate male sterile young maize plants which, when crossed with young plants having a wild-type genotype, in particular with young plants belonging to an elite line, produce seeds heterozygous for the AMS transgene. The elite line used for this cross may in particular be that used for the backcrossing step optionally carried out in the methods above. In this case, the seeds produced can be used to produce commercial seeds derived from a maize hybrid. According to another embodiment, the wild-type elite line can be replaced with a maize hybrid so as to obtain seeds which will serve to produce a three-way hybrid.

According to another aspect of the invention, the method for the production of maize seeds homozygous for the AMS transgene can be used for the production of proteins of interest, in particular therapeutic and/or prophylactic interest. The protein of interest may be produced throughout the plant or else preferentially concentrated in specific organs, such as the seeds.

The method according to the invention has the advantage of making it possible to produce such proteins under controlled and large-scale conditions. It is therefore of great value to the pharmaceutical field.

To do this, a vector containing the barnase gene, conferring male sterility, under the control of the A9 promoter, and a gene of therapeutic and/or prophylactic interest can be constructed. This vector can be used to obtain plants containing the gene conferring male sterility and the gene of therapeutic and/or prophylactic interest, which may then be used according to the system of production as described in order to produce seeds expressing the protein of therapeutic and/or prophylactic interest.

In particular, such a vector can be used to obtain a young maize plant heterozygous for an AMS transgene (and therefore for the gene encoding the protein of interest), which young maize plant can then be crossed with a fertility-restoring young maize plant of genotype (+/+; SSB/+).

Advantageously, the gene of therapeutic and/or prophylactic interest is genetically linked to the AMS transgene.

Such a system makes it possible to produce only the protein of therapeutic and/or prophylactic interest in the seed, for example, or in an organ of the plant, whatever the developmental stage, depending on the regulatory sequence used in the gene encoding said protein of interest, since the protein conferring male sterility is not produced in the organs of the plant. This system thus makes it possible to avoid dissemination of the transgene of therapeutic and/or prophylactic interest via the pollen, since the plants are completely male sterile.

The present invention therefore also relates to a method for the production of a seed heterozygous for an AMS transgene, comprising the crossing of a young maize plant derived from a seed homozygous for an AMS transgene, which can be obtained by one of the methods for the production of a seed homozygous for the AMS transgene described above, with a young maize plant having a wild-type genotype.

According to another embodiment, the method for the production of a seed heterozygous for an AMS transgene is characterized in that one of the methods for the production of a seed homozygous for the AMS transgene described above also comprises the crossing of a young maize plant derived from said seed homozygous for an AMS transgene with a young maize plant having a wild-type genotype.

Fertilization of male sterile young maize plants derived from these seeds, by an unrelated young maize plant having a wild-type genotype, advantageously produces hybrid seeds which benefit from the vigor effect (heterosis).

According to another aspect, the system proposed by the present invention advantageously makes it possible to maintain young maize plants homozygous for an AMS transgene and heterozygous for a fertility-restoring gene linked to a “small seed” phenotype marker. The methods for the production of maize seeds homozygous for the AMS transgene described above in fact comprise a final selection of seeds, in particular on the basis of their normal seed phenotype. The seeds of “small seed” phenotype not selected by these methods can be sown, and the young plants generated can then be self-fertilized, thus producing in particular seeds homozygous for the AMS transgene and heterozygous for the fertility-restoring gene linked to a “small seed” phenotype marker.

The invention therefore relates to a method for the multiplication of a young maize plant homozygous for an AMS transgene and heterozygous for a fertility-restoring gene linked to a “small seed” phenotype marker, comprising the steps consisting in:
a) self-fertilizing young maize plants homozygous for an AMS transgene and heterozygous for a fertility-restoring gene linked to a “small seed” phenotype marker, which can be obtained from a seed from one of the methods for the production of seeds homozygous for an AMS transgene and heterozygous for a fertility-restoring gene linked to a “small seed” phenotype marker described above,
b) selecting seeds homozygous for the AMS transgene and having a “small seed” phenotype,
c) selecting the seeds homozygous for the AMS transgene and heterozygous for a fertility-restoring gene linked to a “small seed” phenotype marker, obtained by self-fertilization of the young maize plants obtained from the seeds obtained according to step b).

Preferably, step b) of the method above comprises densimetric separation, in particular using a densimetric table or column, or a flotation system. A subject of the present invention is also nucleotide constructs, referred to as expression cassettes, comprising a promoter nucleotide sequence functionally linked to at least one gene of interest.

Said gene of interest may also be combined with other regulatory elements such as activators and transcription termination sequences (terminators). Other elements such as introns, enhancers, polyadenylation sequences and derivatives can also be present in the nucleic acid sequence of interest, in order to improve the expression or the functioning of the transforming gene. The expression cassette may also contain 5′ untranslated sequences referred as “leader” sequences. Such sequences can improve translation.

Preferably, in the methods for the production of seeds described above, the young maize plant comprising an AMS transgene conferring artificial nuclear male sterility is characterized in that the AMS transgene, preferably the barnase gene (Hartley, 1988; Gene Bank No. X 12871), is included in an expression cassette, under the control of a promoter specific for pollen generation and of a terminator, and genetically linked to a gene encoding a selection agent under the control of a promoter and of a terminator.

Advantageously, said expression cassette may also comprise a gene encoding a protein of interest.

Preferably, said gene encoding a protein of interest is genetically linked to the AMS gene, in particular the barnase gene. More preferably, the gene encoding a protein of interest is not under the control of said promoter specific for pollen generation.

The promoter specific for pollen generation is in particular a promoter which allows specific expression in the anther, chosen from the group consisting of the A3 promoter (WO 92/11379), the A6 promoter, the A9 promoter (WO 92/11379), corresponding to the 5′ noncoding region of the Arabidopsis thaliana A9 gene, and the anther tapetum-specific promoters such as TA29, TA26, TA13 (WO 89/10396) or Mac2 (WO 00/68403). Among the genes encoding a selection agent (also referred to as selection marker genes), use may in particular be made of genes which confer resistance to an antibiotic (Herrera-Estrella et al., 1983) such as hygromycin, kanamycin, bleomycin or streptomycin, or to herbicides (EP 242 246), such as glufosinate, glyphosate or bromoxynil.

Preferably, said gene encoding a selection agent is chosen from the bar gene (White et al., 1990; Gene Bank No. X 17220) which confers resistance to the herbicide Basta® (glufosinate) and the NptII gene which confers resistance to kanamycin (Bevan et al., 1983).

The excision system for removing the gene encoding a selection agent may be a transposition system, such as in particular the maize Ac/Ds system (WO 02/101061), or a recombination system, such as in particular the P1 bacteriophage Cre/lox system, the yeast FLP/FRT system (Lyzrik et al., 1997), the Mu phage Gin recombinase, the E. coli Pin recombinase or the pSR1 plasmid R/RS system. A cotransformation system (Komari et al., 1996) can also be used. Preferably, the system used will be the maize Ac/Ds system.

According to a preferred embodiment, said gene is included within the Ds transposable element (also called dissociating element or mobilizable sequence of a transposon).

The Ds transposon used is described in the publication by Yoder et al. (1993). The Ds element is an Ac element which has undergone important mutations or deletions in the sequence encoding the transposase. It is able to excise itself from its insertion site only in the presence of an active transposase source Ac. It is therefore Ac-dependent. A preferred system for removing a gene encoding a selection agent can comprise two components:
a first plant having no active transposase, into which a construct comprising the cassette for expression of the gene of interest and that of the gene encoding a selection agent, bordered by the mobilizable sequences of a transposon, can be integrated,
a second plant containing in its genome a gene encoding an endogenous active transposase.

The crossing of these two plants results in the obtaining of regenerants, obtained from F1 plants or from F2 plants selected for the presence of the gene of interest but no gene encoding a selection agent.

Preferably according to the invention, the promoter combined with the gene encoding a selection agent is a constitutive promoter, such as the actin promoter-actinintron, corresponding to the 5′ noncoding region of the rice actin 1 gene and its first intron (McElroy et al., 1991; Gene Bank No. S 44221). The presence of the first actin intron makes it possible to increase the level of expression of a gene when it is fused 3′ of a promoter. This promoter sequence allows, for example, constitutive expression of the bar gene.

Among the terminators which may be used with the AMS transgene or the gene encoding a selection agent, mention may in particular be made of:
the Nos 3′ terminator, nopaline synthase terminator which corresponds to the 3′ noncoding region of the nopaline synthase gene originating from the Ti plasmid of Agrobacterium tumefaciens nopaline strain (Depicker et al., 1982), and
the CaMV 3′ terminator, corresponding to the 3′ noncoding region of the sequence of the circular double-stranded DNA cauliflower mosaic virus producing the 35S transcript (Franck et al. 1980; Gene Bank No. V 00141).

According to another aspect, the present invention relates to an expression cassette comprising a fertility-restoring gene genetically linked to at least one gene encoding a “small seed” phenotype, combined with elements which allow their expression in plant cells, in particular a transcription promoter and terminator.

Among the transcription promoters which can be used in combination with the gene encoding a “small seed” phenotype, mention may be made in particular of:
the HMWG (high molecular weight glutenin) promoter corresponding to the 5′ noncoding region of the wheat (Triticum aestivum) glutenin gene, an albumen storage protein. This seed-specific promoter is described in the publication by Robert et al. (1989),
the B32 promoter, described in the publication by N. Di Fonzo et al. (1988).

Preferably, said expression cassette comprising a fertility-restoring gene genetically linked to at least one gene encoding a “small seed” phenotype is characterized in that said fertility-restoring gene is the barstar gene (Hartley, 1998) placed under the control of a promoter specific for pollen generation, in particular an anther-specific promoter such as pA3, pA6, pA9 or pTA29, or of the Mac2 promoter, and of the CaMV 3′ terminator or Nos 3′ terminator, genetically linked to a gene encoding a selection agent under the control of the actin promoter-actin intron and of the CaMV 3′ terminator or Nos 3′ terminator.

Among the genes encoding a selection agent, use may in particular be made of genes which confer resistance to an antibiotic such as hygromycin, kanamycin, bleomycin or streptomycin, or to herbicides such as glufosinate, glyphosate or bromoxynil. Preferably, said gene encoding a selection agent is chosen from the bar gene which confers resistance to the herbicide Basta® and the NptII gene which confers resistance to kanamycin.

Preferably, the gene encoding a “small seed” phenotype is chosen from the shrunken 2 and brittle 2 genes in antisense orientation.

According to another aspect, the invention relates to a vector, in particular a plasmid, characterized in that it contains at least one expression cassette as described above.

The invention also relates to a cellular host, in particular a bacterium such as Agrobacterium tumefaciens, transformed with said vector. Such a cellular host is used for transfecting maize cells with a vector according to the invention.

The invention therefore also relates to a maize cell transformed with at least one vector as described above. The transformation of plant cells can be carried out by transfer of the abovementioned vectors into the protoplasts, in particular after incubation of the latter in a solution of polyethylene glycol (PG) in the presence of divalent cations (Ca²⁺) according to the method described in the article by Krens et al. (1982).

The transformation of the plant cells can also be carried out by electroporation, in particular according to the method described in the article by Fromm et al. (1986).

The transformation of the plant cells can also be carried out using a gene gun for projecting, at very high speed, metal particles covered with DNA sequences of interest, thus delivering genes into the cell nucleus, in particular according to the technique described in the article by Finer et al. (1992).

Another method for transforming the plant cells is that of cytoplasmic or nuclear microinjection.

According to a particularly preferred embodiment of the method of the invention, the plant cells are transformed with a vector according to the invention, said cellular host being capable of infecting said plant cells, allowing the integration into the genome of the latter of the DNA sequences of interest initially contained in the genome of the abovementioned vector.

Advantageously, the abovementioned cellular host used is “Agrobacterium tumefaciens”, in particular according to the methods described in the articles by Bevan (1984) and by An et al. (1986), or else Agrobacterium rhizogenes, in particular according to the method described in the article by Jouanin et al. (1987).

Preferably, the transformation of the plant cells is carried out by transfer of the T region of the tumor-inducing extrachromosomal circular plasmid Ti of Agrobacterium tumefaciens, using a binary system (Watson et al., 1994).

To do this, two vectors are constructed. In one of these vectors, the T-DNA region has been removed by deletion, with the exception of the right and left edges, a marker gene being inserted between them so as to allow selection in the plant cells. The other partner of the binary system is an auxiliary Ti plasmid, which is a modified plasmid that no longer has any T-DNA but still contains the vir virulence genes required to transform the plant cell. This plasmid is maintained in Agrobacterium.

An object of the present invention is also to produce transgenic young maize plants, parts of a plant or plant extracts, characterized in that they are regenerated from the transformed plant cell.

The invention relates in particular to a fertility-restoring young maize plant, characterized in that it comprises in its genome a fertility-restoring gene linked to a “small seed” phenotype marker, or a young maize plant homozygous for an AMS transgene and heterozygous for a fertility-restoring gene linked to a “small seed” phenotype marker, obtained from a maize seed homozygous for an AMS transgene and heterozygous for a fertility-restoring gene linked to a “small seed” phenotype marker.

The invention is also directed toward a kit for implementing a method for the multiplication of a young maize plant homozygous for an AMS transgene and heterozygous for a fertility-restoring gene linked to a “small seed” phenotype marker described above, characterized in that it comprises maize seeds homozygous for an AMS transgene and heterozygous for a fertility-restoring gene linked to a “small seed” phenotype marker, and oligonucleotides specific for the AMS transgene that are useful as primers for detecting, by PCR, the seeds homozygous for an AMS transgene and heterozygous for a fertility-restoring gene linked to a “small seed” phenotype marker.

The examples and figures below illustrate the invention without however limiting the scope thereof:

### FIGURE LEGENDS

FIG. 1 represents the diagram of the principle of a succession of steps resulting in the production of a hybrid seed according to the invention.

FIG. 2 represents the plasmid pRec 274 comprising the barnase gene, conferring male sterility, and the bar gene, conferring resistance to the herbicide Basta®.

FIG. 3 represents the donor plasmid pBIOS 274 comprising the spectinomycin-resistance gene and also the T-DNA carrying the barnase gene, under the control of the A9 promoter, and the bar gene, under the control of the rice actin promoter.

FIG. 4 represents the donor plasmid pBIOS 424 comprising the A9 promoter-barnase-CaMV 3′ terminator and the kanamycin-resistance gene NptII in a Ds dissociating element.

FIG. 5 represents the plasmid p3222 comprising the antisense sequence of the brittle 2 gene and also the fertility-restoring barstar gene.

FIG. 6 represents the plasmid p3223 comprising the antisense sequence of the shrunken 2 gene and also the fertility-restoring barstar gene.

FIG. 7 represents the plasmid p4962 comprising the antisense sequences of the shrunken 2 and brittle 2 genes and also the fertility-restoring barstar gene.

FIG. 8 represents the plasmid pDM 302 comprising the expression cassette for the bar gene.

FIG. 9 represents the donor plasmid pBIOS 273 which contains an expression cassette comprising the rice actin promoter, the bar gene and the Nos 3′ terminator.

### EXAMPLES

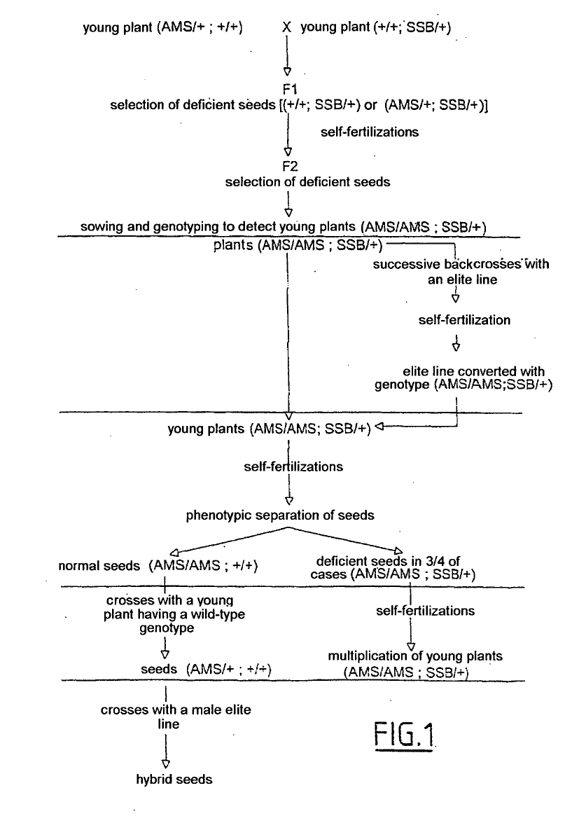

A nonlimiting illustration of one of the methods according to the present invention is described in FIG. 1.

The construction of the various plasmids and the ligation thereof, and the transformation of Escherichia coli XLI blue bacteria made competent beforehand, are carried out by means of the usual recombinant DNA techniques (Sambrook et al., 1989).

The construction of the expression vectors and the transformation techniques used are within the scope of those skilled in the art following standard techniques.

### Example 1

### AMS Construct

1.a) Construction of a Plasmid (pRec 274) Comprising the Barnase Gene, Conferring Male Sterility, Linked to the Bar Gene, Conferring Resistance to the Herbicide Basta®

The vector used for transforming the maize with Agrobacterium tumefaciens is in the form of a superbinary plasmid of approximately 50 kb (pRec 274).

The superbinary vector used for the transformation contains:
an ori region: Col E1 plasmid origin of replication, necessary for maintenance and multiplication of the plasmid Escherichia coli. This origin of replication is not functional in Agrobacterium tumefaciens,
an origin of replication that is functional in Agrobacterium tumefaciens and Escherichia coli,
the cos region of the lambda bacteriophage, which may be of use for manipulating the vector in vi tro,
the additional virB, virC and virG regions of Agrobacterium tumefaciens which increase the transformation efficiency,
genes for resistance to tetracycline (Tetra) and to spectinomycin (Spect), which are expressed only in the bacteria,
a T-DNA carrying the barnase gene conferring male sterility and the bar gene conferring resistance to the herbicide Basta®, these two genes being functionally linked to elements which allow their transcription. In the present example, the barnase gene is under the control of the A9 promoter and of the CaMV 3′ terminator, the bar gene being under the control of the rice actin promoter-actin intron and of the Nos 3′ terminator.

The Streptomyces hygroscopicus bar gene encodes a phosphinothricin acyl transferase (PAT) which detoxifies phosphinothricin (Basta® herbicide selection agent) by acetylation (White et al., 1990). It is generally used to select transformed plants which contain both the gene of interest and this gene encoding a selection agent, and which are therefore resistant to the herbicide.

The barnase gene, which confers male sterility, encodes a ribonuclease (RNase). This gene was isolated from Bacillus amyloliquefasciens and is described in the publication by Hartley (1988).

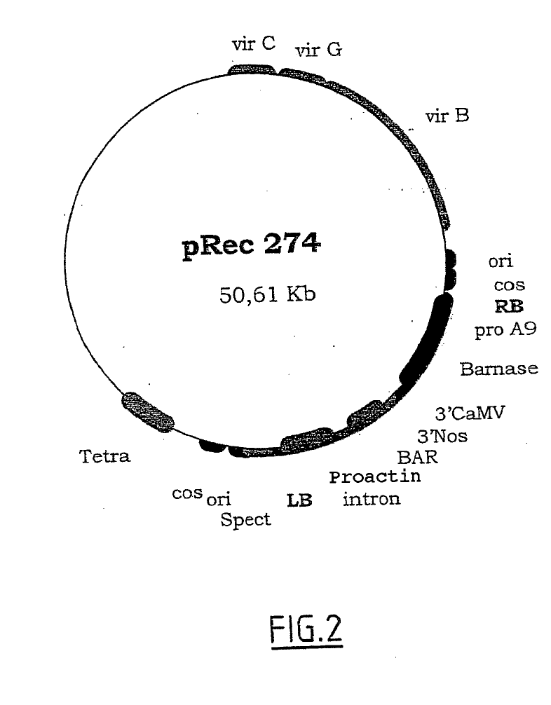

The superbinary plasmid pRec 274 is represented in FIG. 2.

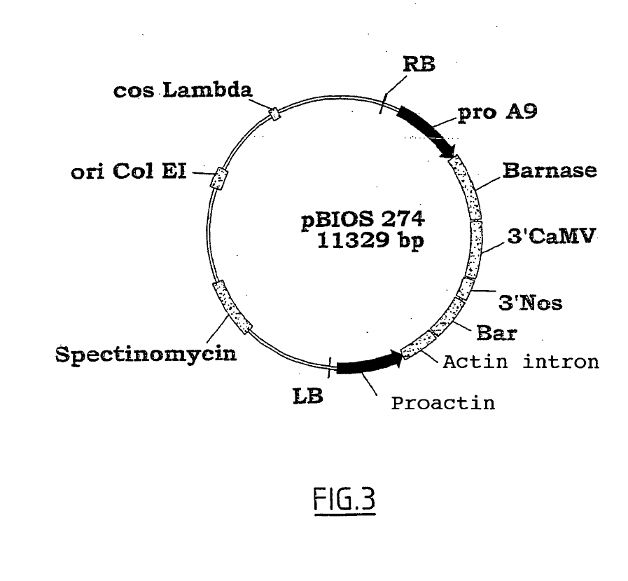

This superbinary vector is attained by homologous recombination of an acceptor plasmid pSB1 (EP 672 752), derived from an Agrobacterium tumefaciens Ti plasmid, with a donor plasmid pBIOS 274 (FIG. 3), derived from pUC (Messing, 1983).

The donor plasmid possesses the spectinomycin-resistance gene and also the T-DNA carrying the barnase gene, under the control of the A9 promoter, and the bar gene, which is also a selection gene, under the control of the rice actin promoter.

The donor and acceptor plasmids possess a region of homology that is sufficient to allow homologous recombination and to obtain the “superbinary” vector.

The technique of transformation with Agrobacterium tumefaciens allows integration of only the T-DNA consisting of the right border (RB) and left border (LB) framing the gene of interest (barnase) and the gene encoding a selection agent.

1.b) Construction of a Plasmid (pRec 424) Comprising the Barnase Gene Conferring Male Sterility Linked to the NPT-II Gene Conferring Kanamycin Resistance

The vector used for transforming the maize with Agrobacterium tumefaciens is in the form of a superbinary plasmid of approximately 50 kb (pRec 424).

The superbinary vector used for the transformation contains:
an ori region: Col E1 plasmid origin of replication, necessary for maintenance and multiplication of the plasmid Escherichia coli. This origin of replication is not functional in Agrobacterium tumefaciens,
an origin of replication that is functional in Agrobacterium tumefaciens and Escherichia coli,
the cos region of the lambda bacteriophage, which may be of use for manipulating the vector in vitro,
the additional virB, virC and virG regions of Agrobacterium tumefaciens which increase the transformation efficiency,
genes for resistance to tetracycline (Tetra) and to spectinomycin (Spect), which are expressed only in the bacteria,
a T-DNA carrying the barnase gene, conferring male sterility, and NptII gene inserted into a Ds element, conferring kanamycin resistance, these two genes being functionally linked to elements which allow their transcription. In the present example, the barnase gene is under the control of the A9 promoter and of the CaMV 3′ promoter, the NptII gene inserted into a Ds element being under the control of the actin promoter and of the Nos 3′ terminator.

The NptII gene was isolated from the Escherichia coli Tn5 transposon (Berg et al., 1983). This gene encodes the neomycin phosphotransferase type II enzyme which catalyzes the O-phosphorylation of aminoglycoside antibiotics such as neomycin, kanamycin, gentamycin and G418 (Davies and Smith, 1978). This gene confers kanamycin resistance, which is used as a selection agent in plant genetic transformation. It is described by Bevan et al. (Genbank No. U00004).

The barnase gene, which confers male sterility, encodes a ribonuclease as mentioned in Example 1.a above.

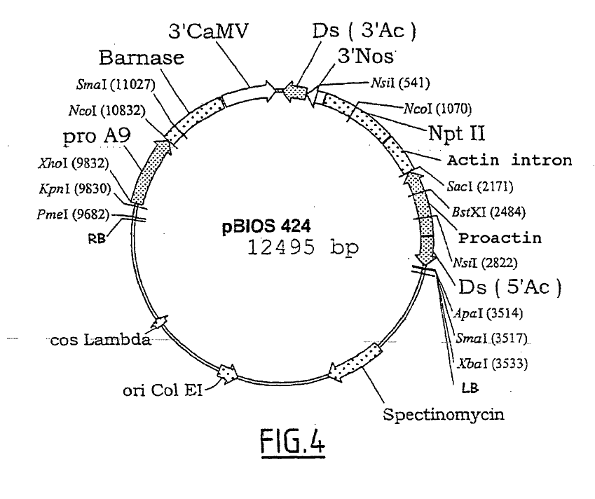

This superbinary vector is obtained by homologous recombination of an acceptor plasmid pSB1 (EP 672 752), derived from an Agrobacterium tumefaciens Ti plasmid, with a donor plasmid pBIOS 424 (FIG. 4), derived from pUC (Messing, 1983).

The donor plasmid pBIOS 424 possesses the spectinomycin-resistance gene and also the T-DNA carrying the barnase gene under the control of the A9 promoter and the NptII gene placed under the control of the actin promoter, inserted into a Ds element.

The donor plasmid pBIOS 424 (A9 promoter-barnase-CaMV 3′ terminator and the kanamycin-resistance gene NptII in a Ds dissociating element) was generated in the following way:

The fragment containing the A9 promoter-barnase gene-CaMV 3′ terminator cassette was isolated by:
a) restriction with XhoI,
b) treatment with T4 DNA polymerase so as to generate blunt ends, and
c) restriction with XbaI using the plasmid pBIOS 274 (FIG. 3).

This fragment (XhoI/blunt-XbaI) was then introduced into the vector pBIOS 415 (described below) opened by means of the EcoRV and XbaI restriction enzymes.

The EcoRV-XbaI fragment thus generated contains the plasmid sequences of the vector pSB12 (Japan Tobacco, EP 672 752) and the cassette Ds element-actin promoter-actin intron-NPTII gene-Nos terminator-Ds element.

The plasmid pBIOS 415 contains the GFP gene under the control of the CsVMV promoter (WO 97/48819) and of the Nos terminator (XbaI-XhoI fragment) in the acceptor vector pBIOS 340. The vector pBIOS 340 is a vector containing the plasmid sequences of the vector pSB12 (Japan Tobacco, EP 672 752) and the cassette Ds element-actin promoter-actin intron-NPTII gene-Nos terminator-Ds element.

The donor and acceptor plasmids possess a region of homology that is sufficient to make it possible to obtain the “superbinary” vector by homologous recombination.

The technique of transformation with Agrobacterium tumefaciens allows integration of only the T-DNA consisting of the right border (RB) and left border (LB) framing the gene of interest (barnase gene) and the gene encoding a selection agent.

### Example 2

### Construct Restorer-Phenotypic Marker Linked to Seed Size

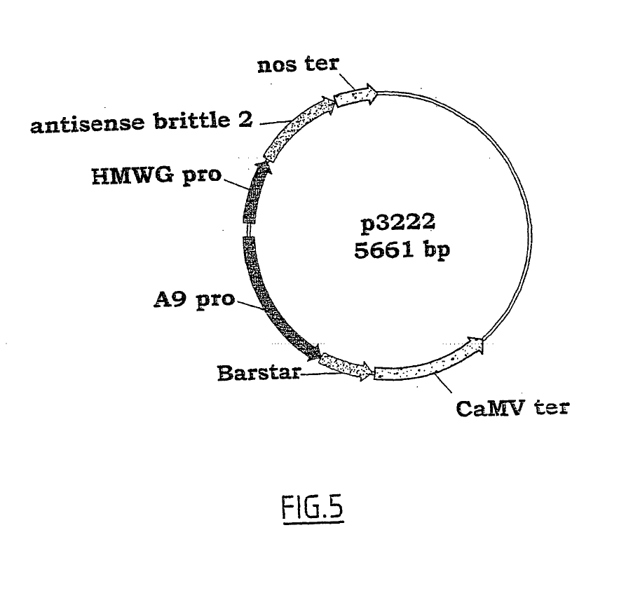

2.a) Construction of a Plasmid (p3222, FIG. 5) Comprising the Antisense Sequence of the Brittle 2 Gene and also the Fertility-Restoring Barstar Gene

The brittle 2 gene encodes a subunit of ADP glucose pyrophosphorylase, an enzyme involved in starch synthesis. In the antisense orientation, it makes it possible to inhibit the synthesis of this subunit, which produces a mutant phenotype in which the seed size is 50% less than the normal size.

The barstar gene encodes a barnase-specific inhibitor. It was isolated from Bacillus amyloliquefaciens and is described in Hartley (1998) (Genbank No. X15545).

The plasmid p3222 carries two individually cloned expression cassettes. The first cassette comprises the HMWG promoter, the brittle 2 gene in antisense orientation and the Nos terminator. The second cassette comprises the A9 promoter, the barstar gene and the CaMV terminator.

The brittle 2 gene was synthesized by PCR from maize albumen cDNA with the oligonucleotides Bt5 (CCGGATCCATGTGACAGACAGTGTTA, SEQ ID No. 1) containing a BamHI site, and Bt3 (AAGCCCGGGACTTGTACTAACTGTTTC, SEQ ID No. 2) containing a SmaI site.

The 600 bp PCR fragment thus obtained was digested with SmaI and BamHI and cloned between the HMWG promoter and the nos terminator region of the plasmid p3214 opened with SmaI and BamHI. The plasmid p3215 thus obtained contains the HMWG promoter-brittle 2 (antisense orientation)-nos expression cassette.

A 270 bp fragment containing the barstar gene was amplified by PCR from the plasmid pWP127 with the oligonucleotides BPR5 (TATCGGATCCAAATCATAAGAAAGGAG, SEQ ID No. 3) containing a BamHI site, and BPR4 (GAAGATCTATATTGTTCATCCCATTG, SEQ ID No. 4) containing a Bg1II site. The PCR fragment thus obtained was digested with BamHI and Bg1II and cloned between the A9 promoter and the CaMV terminator region of the plasmid p1415 opened with BamHI. The plasmid p3072 thus obtained contains the barspar genes under the control of the A9 promoter and of the CaMV terminator region. The plasmid p3222 according to Example 2.a) corresponds to the insertion of the cassette HMWG promoter-brittle 2 (antisense orientation)-nos (KpnI-SacI fragment) of p3215 into p3072 opened with KpnI and SacI.

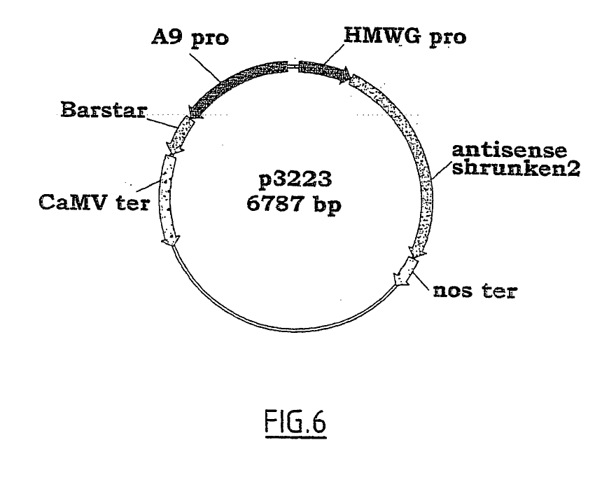

2.b) Construction of a Plasmid (p3223, FIG. 6) Comprising the Antisense Sequence of the Shrunken 2 Gene and also the Fertility-Restoring Barstar Gene

The shrunken 2 gene encodes the other subunit of ADP glucose pyrophosphorylase, an enzyme involved in starch synthesis. In the antisense orientation, it makes it possible to inhibit the synthesis of this subunit, which produces a mutant phenotype in which the seed size is 40% less than the normal size.

The plasmid p3223 carries two individually cloned expression cassettes. The first cassette comprises the HMWG promoter, the shrunken 2 gene in antisense orientation and the Nos terminator. The second cassette comprises the A9 promoter, the barspar gene and the CaMV terminator.

The shrunken 2 gene was synthesized by PCR from maize albumen cDNA with the oligonucleotides New Sh5 (GCACCCGGGAGGAGATATGCAGTTTG, SEQ ID No. 5) containing an SmaI site, and Sh3 (GACTGCAGCACAAATGGTCAAG, SEQ ID No. 6) containing a PstI site.

The 1800 bp PCR fragment thus obtained was digested with SmaI and PstI and cloned between the HMWG promoter and the nos terminator region of the plasmid p3214 opened with SmaI and PstI. The plasmid p3217 thus obtained contains the HMWG promoter-shrunken 2 (antisense orientation)-nos expression cassette.

A 270 bp fragment containing the barstar gene was amplified by PCR from the plasmid pWP127 with the oligonucleotides BPR5 (TATCGGATCCAAATCATAAGAAAGGAG, SEQ ID No. 7), containing a BamHI site, and BPR4 (GAAGATCTATATTGTTCATCCCATTG, SEQ ID No. 8) containing a Bg1II site. The PCR fragment thus obtained was digested with BamHI and Bg1II and cloned between the A9 promoter and the CaMV terminator region of the plasmid p1415 opened with BamHI. The plasmid p3072 thus obtained possesses the barstar gene under the control of the A9 promoter and of the CaMV terminator region.

The plasmid p3223 according to Example 2.b) corresponds to the insertion of the cassette HMWG promoter-shrunken 2 (antisense orientation)-nos (KpnI-SacI fragment) of p3217 into p3072 opened with KpnI and SacI.

2.c) Construction of a Plasmid (pRec 4962) Comprising the Antisense Sequences of the Shrunken 2 and Brittle 2 Genes and also the Fertility-Restoring Barstar Gene

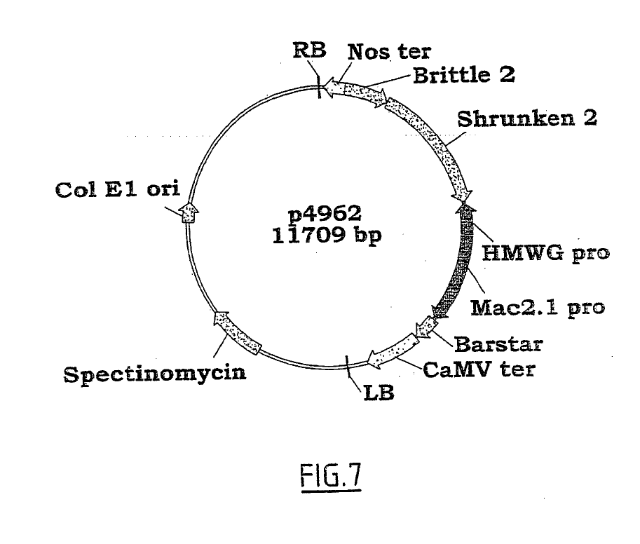

The plasmid p4962 (FIG. 7) carries two individually cloned expression cassettes, the first cassette comprising the HMWG promoter, the shrunken 2 gene in antisense orientation, the brittle 2 gene in antisense orientation and the Nos terminator and the second cassette comprising the Mac2.1 promoter, the barstar gene and the CaMV terminator. This plasmid was constructed by means of conventional molecular biology techniques known to those skilled in the art.

The plasmid p4962 is in the form of a donor vector derived from the vector pSB12 (Japan Tobacco, EP 672 752) of approximately 11.7 kb, comprising:
an ori region: Col E1 plasmid origin of replication, necessary for the maintenance and the multiplication of the plasmid in the bacterium,
a spectinomycin-resistance gene which is expressed only in the bacteria,
a T-DNA comprising the male fertility-restoring gene (barstar gene) and the antisense sequences of the shrunken 2 and brittle 2 genes conferring a “small seed” phenotype.

In the present example, the barstar gene is under the control of the Mac2.1 promoter and of the CaMV 3′ terminator, the shrunken 2 and brittle 2 genes in the antisense orientation being under the control of the HMWG promoter and of the Nos 3′ terminator. The superbinary vector pRec 4962 is obtained by homologous recombination of an acceptor plasmid pSB1 (Japan Tobacco, EP 672 752), derived from an Agrobacterium tumefaciens Ti plasmid, with the donor plasmid p4962.

### Example 3

### Selection Plasmid

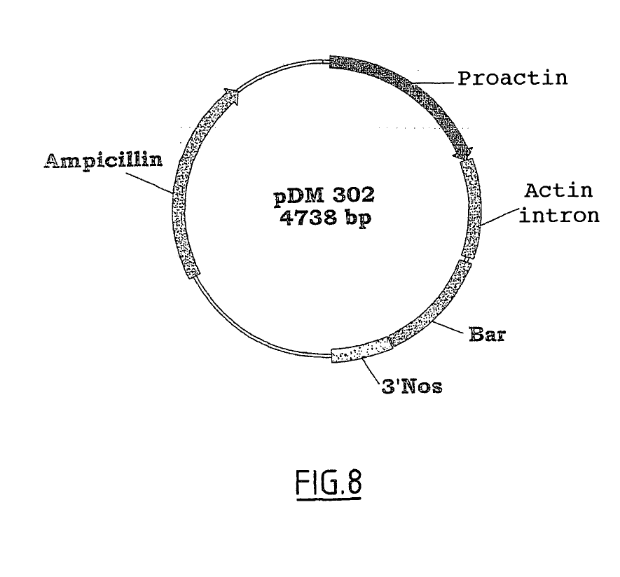

3.a) Construction of a Plasmid (pDM 302, FIG. 8) Comprising the Bar Gene Conferring Resistance to the Herbicide Basta® used as Selection Plasmid in Cotransformation with the Restorer Plasmid

As indicated in Example 1, the bar gene makes it possible to select the transformed plants which are resistant to the herbicide Basta®.

The plasmid pDM302 (Cao et al., 1992) carries the expression cassette comprising the actin promoter-actin intron, the bar gene and the Nos terminator.

This plasmid pDM302 was obtained in the following way:

The coding region of the Streptomyces hygroscopicus bar gene encoding PAT (phosphinothricin acetyl transferase) activity was excised from the plasmid pIJ4104 (White et al., 1990) by means of the SmaI restriction enzyme (600 bp fragment) and cloned into the expression vector pCOR113 (McElroy et al., 1991) behind the 5′ fragment (promoter and first intron) of the rice actin 1 gene (Act-1). This generated the 4.9 kb plasmid pDM301 containing the Act1-bar expression cassette. The Act1-bar expression cassette of pDM301 was excised as a 2.0 kb XhoI-XbaI restriction fragment and cloned between the SalI and XbaI sites upstream of the terminator sequence of the nos gene encoding nopaline synthase (plasmid pNOS72). The 4.7 kb pDM302 plasmid thus obtained contains the Act1-bar-nos expression cassette.

3.b) Construction of a Plasmid pRec 273 Comprising the Bar Gene Conferring Resistance to the Herbicide Basta® used as a Selection Plasmid in Cotransformation with the Restorer Plasmid

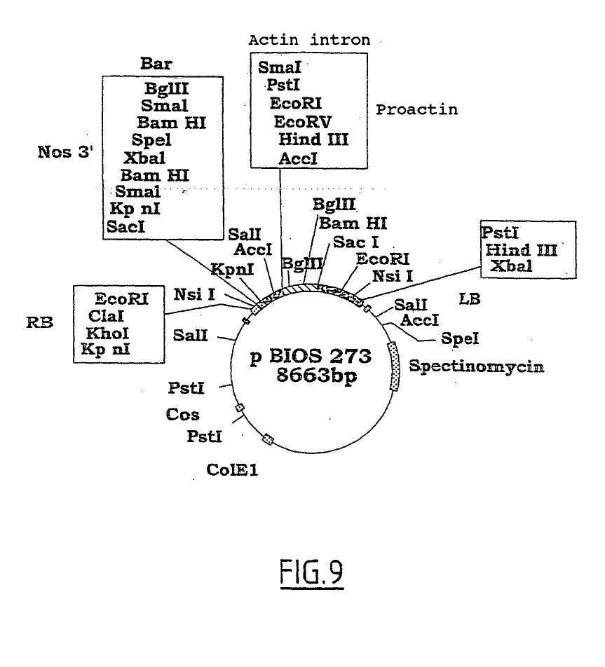

The plasmid pBIOS 273 (FIG. 9) carries an expression cassette comprising the rice actin promoter, the bar gene and the Nos 3′ terminator. This plasmid was constructed by means of conventional molecular biology techniques known to those skilled in the art.

The plasmid pBIOS 273 is in the form of a donor vector derived from the vector pSB12 (Japan Tobacco, EP 672 752), of approximately 8.6 kb, comprising:
an ori origin: Col E1 plasmid origin of replication, necessary for the maintenance and the multiplication of the plasmid in the bacterium,
a spectinomycin-resistance gene which is expressed only in the bacteria,
a T-DNA comprising the bar gene conferring resistance to the herbicide Basta® under the control of the rice actin promoter and of the Nos 3′ terminator.

The plasmid pBIOS 273 was generated in two steps:
cloning of the BspDI/XhoI fragment (actin promoter-bar gene-Nos terminator) of the vector pDM 302 (Cao et al., 1992) into the SmaI and BspDI sites of the vector pSB12 (Japan Tobacco, EP 672 752). The vector resulting from this cloning is called pBIOS 272.
delection of the XhoI site at position 3363 of the vector pBIOS 272 by partial digestion with XhoI and the action of DNA polymerase I, large (Klenow) fragment. The vector obtained, which has a unique XhoI site, is called pBIOS 273.

The superbinary vector pRec 273 is obtained by homologous recombination of an acceptor plasmid pSB1 (Japan Tobacco, EP 672 752), derived from an Agrobacterium tumefaciens Ti plasmid, with the donor plasmid pBIOS 273.

### Example 4

### Production of a Male Sterile Maize Line Heterozygous for the AMS Transgene

4.a) Production of a Male Sterile Maize Line Heterozygous for the AMS Transgene (AMS/+) by Transformation of Maize with the Plasmid Described According to Example 1a (pRec 274)

A heterozygous male sterile maize line expressing the barnase (conferring male sterility) and bar (glufosinate resistance) genes respectively under the control of the A9 (Paul et al., 1992) and actin-intron (McElroy et al., 1991) promoters is obtained by transformation with Agrobacterium tumefaciens according to the method described by Ishida et al. (1996).

In the following example, the heterozygous (AMS/+) male sterile maize line was produced by the Agrobacterium tumefaciens transformation method. Other transformation techniques known to those skilled in the art may be used.

Obtaining and Preparation of the Plant Material:

Maize ears are decontaminated for 15 to 20 minutes in 20% Domestos with stirring and are then rinsed with sterile water before removing the immature embryos, which are placed in LSinf medium. The optimal size of the immature embryos is from 1 to 1.2 mm, which corresponds to 10+/−2 days after fertilization. The embryos are then vortexed, the LSinf medium is removed, and rinsing in LSinf medium is carried out before vortexing again.

Preparation of the Bacteria:

Agrobacterium tumefaciens bacteria (strain LBA 4404) containing the superbinary plasmid pRec 274 (as described in Example la) are placed in culture in YP medium supplemented with a selective agent suitable for the strain. 2 to 3 days later, the bacteria are suspended in LSinf medium+100 μM acetosyringone. The concentration of the inoculum is considered to be at 1×10⁹ bacteria/ml.

Inoculation and Coculturing:

After having removed the LSinf medium, the embryos are brought into contact with the agrobacteria. After having vortexed, 50 μl of 1% Pluronic F68 are added and incubation is carried out for 5 minutes at ambient temperature. The inoculum is removed and the embryos are recovered and placed on 1.5LSA medium, scutellum facing upwards. After having sealed the dish, incubation is carried out at 25° C. in the dark for 5 days.

Selection of Transformed Calluses:

At the end of the coculturing, the embryos are transferred onto LSD5 medium and placed in a proportion of 25 per dish sealed with Urgopore. Incubation is carried out at 25° C. in the dark for 2 weeks (1^{st} selection). A 2^{nd} selection consists in transferring the embryos onto LSD10 medium, by cutting the germinations. Incubation is carried out for 3 weeks under the same conditions as in the 1^{st} selection. A 3^{rd} selection is carried out by excising the “good” type I calluses so as to obtain fragments of 1-2 mm. They are placed in culture on LSD10 medium, followed by incubation for 3 weeks under the same conditions as in the first and second selections.

Regeneration of Transformed Plantlets:

The type I calluses which have proliferated are placed on LSZ2 medium and the dishes are sealed with scellofrais® and placed in a culture chamber at 27° C. for 2 weeks. The type I calluses which have proliferated are again collected, separated and placed on RM+G4C100 medium. The dishes are sealed with scellofrais® and placed in a culture chamber at 27° C. The regenerated plantlets are subcultured on T1G4 medium and placed under continuous light at 27° C. for one to two weeks. The plantlets which have attained a sufficient development are transferred to a phytotron.

In order to identify the plantlets resistant to the herbicide Basta®, and which have therefore integrated the transgene, a selection step is carried out with a Basta F1 solution (AgrEvo France). This solution is applied by leaf painting on maize plants at the 4- to 5-leaf stage. The ammonium glufosinate concentration in the treating solution is 0.75 grams per liter.

The resistant plants exhibit a non-necrosed region 5 days after application of the herbicide. The sensitive plants exhibit necrosis in the treated region; death of the chlorophyll-containing tissues is then observed.

The plants thus regenerated are acclimatized and then cultivated under glass, where they can be crossed or self-fertilized.

4.b) Production of a Male Sterile Maize Line Heterozygous for the AMS Transgene (AMS/+) by Transformation of Maize with the Plasmid Described According to Example 1b (pRec 424)

A heterozygous male sterile maize line expressing the barnase gene conferring male sterility, under the control of the A9 promoter (Paul et al., 1992), is obtained by transformation with A. tumefaciens according to the method described by Ishida et al. (1996). The Agrobacterium tumefaciens transformation technique, with no implied limitation, used in this example is identical to that used in Example 4.a.

Transformation with the expression cassette A9-barnase-CamV 3′-Ds::NPTII comprising the barnase gene under the control of the A9 promoter and of the CaMV 3′ terminator (according to Example lb) on a vector which can be used in transformation via Agrobacterium and which comprises the “kanamycin” selection marker within the Ds transposable element has the advantage of eliminating the marker gene, which will no longer be in the transformed maize line.

In the present example, the plantlets which have integrated the transgene are identified in the following way:

Regeneration of Transformed Plantlets:

The type I calluses which have proliferated are placed on LSZ2 medium and the dishes are sealed with scellofrais® and placed in a culture chamber at 27° C. for 2 weeks. The type I calluses which have proliferated are again collected, separated and placed on RM+G4C100 medium. The dishes are sealed with scellofrais® and placed in a culture chamber at 27° C. The regenerated plantlets are subcultured on T1G4 medium and placed under continuous light at 27° C. for one to two weeks. The plantlets which have attained a sufficient development are transferred to a phytotron.

In order to identify the plantlets resistant to kanamycin and which have therefore integrated the transgene, a selection step (by the cornet drop test) is carried out with a solution of kanamycin at a concentration of 500 mg/l, to which 1% of Tween has been added. 2 to 3 drops of this solution are applied to the maize plants at the 4- to 5-leaf stage.

The plants are analyzed 5 days after application of the kanamycin. The sensitive plants exhibit the appearance of whitish regions (death of the chlorophyll-containing tissues). The resistant plants do not exhibit the appearance of whitish regions 5 days after application of the kanamycin.

The plants thus regenerated are acclimatized and then cultivated under glass, where they may be crossed or self-fertilized.

Once the transformants (comprising the A9-barnase-CaMV 3′-Ds::NPTII expression cassette) have been isolated by the action of the selection agent and/or by molecular analyses, the gene encoding the selection agent is eliminated. The selected transformants are therefore crossed with an active transposase source Ac.

The fertilization is carried out manually using a technique known to those skilled in the art by depositing the pollen from the transposase source Ac onto the bristles of the transformants, preferably in the direction of the male plant possessing the transposase into the female plant containing the Ds element::NptII.

The F1 seeds are germinated so as to obtain plantlets. The plantlets are evaluated for their kanamycin resistance (the resistant plants are conserved) and a PCR test is carried out to detect the somatic excisions (a somatic excision does not generally affect the gametes and results in the formation of chimeric seeds and individuals in which most of the cells still possess the gene encoding the selection agent). The primers used for this PCR test which makes it possible to search for somatic excisions on the F1 plants resistant to the selection agent are as follows:

| | | |
|---|---|---|
| Name | Size (bp) | Sequence |

| | | | |
|---|---|---|---|
| Barn5 | 21 | 5′-GGTTTCGCTCATGTGTTGAGC-3′ | (SEQ ID No. 15) |
| EM11 | 25 | 5′-CATTGCGGACGTTTTTAATGTACTG-3′ | (SEQ ID No. 16) |

The pair of primers Barn5/EM11 makes it possible to visualize the excision (other appropriate primers can also be used). The amplification is different depending on whether or not excision has taken place. The F1 plants in which somatic excision has taken place are therefore selected and then crossed with a plant having a wild-type genotype (WT). The F2 plants are screened in order to identify the plants with the gene of interest without the gene encoding the selection agent (a germinal excision affects the gametes and results in the formation of seeds and of individuals consisting of cells which no longer possess the gene encoding the selection agent).

4.c) Molecular Characterization of the Transformants

The Southern methodology (1975) is used to demonstrate the insertion of the transgene into the genome of the plant and to evaluate the number of copies and to characterize the integration profile.

The genomic DNA of the plants (8 μg) is extracted from the leaves of the plants according to a CTAB (cetyltrimethylammonium bromide) extraction protocol, according to the protocol of J. Keller (DNAP 6701 San Pablo Ave Oakland Calif. 94608 USA) modified by I. Bancroft (Department of Molecular Genetics, Cambridge Laboratory, John Innes Center for Plant Science Research, Colney lane, Norwich, England). The DNA obtained was digested with various restriction enzymes, separated by agarose gel electrophoresis and transferred onto hybond N+nylon membrane and then hybridized with radioactive probes.

The Bar probe for the molecular analyses is prepared in the following way: the plasmid pDM302 (described in Example 3a) of the present invention) is digested with the SmaI enzyme. The 0.6 kb fragment of interest is recovered after migration of the digestion product on electrophoresis gel and purification with the Gene Clean kit (Bio 101, Ozyme). After P32-labeling of 30 ng of fragment, said fragment is used as probe for hybridizing the various blots.

The male sterile transformation event (STB27b) is a transformant obtained by transformation via Agrobacterium tumefaciens according to Example 4a) described above, and which has a single-copy insertion of the T-DNA as described according to Example la. The results from hybridization of the DNA digested with various restriction enzymes (NcoI, SpeI, EcoR V, HindIII and Ecor I), and then hybridized with the Bar probe, show that a single fragment is revealed whatever the enzyme used. The size of the fragment revealed by the HindIII digestion is 1.7 kb. This same type of molecular characterization is carried out for the transformants obtained by transformation by Agrobacterium tumefaciens according to Example 4b).

### Example 5

### Production of a Fertility-Restoring Maize Line Heterozygous for the Fertility-Restoring Gene (SSB/+)

5.a) Production of a Fertility-Restoring Maize Line (SSB/+) Heterozygous for the Fertility-Restoring Gene by Cotransformation of Maize with the Plasmids of Examples 2a, 2b (Restorer Plasmids) and 3a) (Selection Plasmid pDM 302)

A method of genetic transformation which results in the stable integration of the modified genes into the plant's genome is preferably used. This method is based on the use of a particle gun. However, other transformation techniques known to those skilled in the art may be used.

Production of Immature Embryos:

It consists in the self-fertilization of a plant of the HiII line or of the “brother-sister” (sib) crossing of 2 plants of the HiII line. The fertilization is carried out on a single date after having isolated the reproductive organs.

Ear Removal:

The ears are removed when the immature embryos have reached a size of 1.5 mm to 2 mm, i.e. 10 to 11 days after fertilization under our cultivation conditions (temperature of 25° C. during the day and 18° C. at night, 16/8 photoperiod).

Disinfection:

The harvested ears have their spathes and their bristles removed and are then disinfected with 20% (v/v) Domestose for 15 minutes with stirring. The ears are rinsed three times with sterile water.

Embryo Extraction:

The upper part of the seed is cut so as to reveal the albumen, and then a slight pressure on the seed makes it possible to free the albumen. The immature embryo which is still in the seed (against the pericarp) is extracted and then placed on the callogenesis medium N6P6, flat side placed on the agar. Thirty-six embryos per dish are placed in culture for 4 days in a culture chamber at 26° C. and in the dark. This is the initiating period.

Genetic Transformation:

Preparation of the Embryos:

After the initiating period, the embryos are placed, 4 hours before the firing, on the 0.4 M N6P6 osmotic shock medium and are arranged in a proportion of 36 in a small square 2 cm² at the center of the dish. The dishes are sealed with scellofrais and incubated in a culture chamber (26° C. in the dark).

The plasmids carrying the genes to be introduced (plasmids described in Examples 2a, 2b and 3a) are purified on a Qiagen® column according to the manufacturer's instructions. They are then precipitated onto particles of tungsten (M10) according to the protocol described by Klein (1987). The particles thus coated are projected onto the target cells using a gun and according to the protocol described by J. Finer (1992).

The dishes of embryos thus bombarded are then sealed with scellofrais® and cultivated in the dark at 27° C. The first subcultivation takes place 24 h later, and then every fifteen days for 3 months on medium identical to the initiating medium, supplemented with a selective agent, the nature and the concentration of which may vary according to the gene used. The selective agents which can be used generally consist of active compounds of certain herbicides (Basta®, Round up®) or certain antibiotics (hygromycin, kanamycin, etc.). Preferably, the gene for resistance to the herbicide Basta® will be used.

Maturation and Regeneration of Type II Calluses:

When there is sufficient material for an event, it is transferred onto the MM+G2 maturation medium which promotes the development of somatic embryos. The callus is plated out at the surface of the MM+G2 medium. The dishes are placed in a culture chamber at 26° C. in the dark. After 15 days (minimum) on the MM+G2 medium, somatic embryos appear. The latter are subcultivated on RM+G2 regeneration medium (20 to 25 per dish) and grown at 28° C. under light (16 h/24 h). It is considered that 4 regeneration dishes are sufficient to obtain regenerants.

After approximately 15 days on the RM+G2 medium, the embryos have developed into plantlets which are then subcultivated in tubes on the T1+G2 rooting medium. It is considered that 5 plantlets per event are necessary to achieve the acclimatization in a phytotron and the start-ups. These plantlets are placed in a culture chamber under light.

After 3 months, or sometimes earlier, calluses are obtained, the growth of which is not inhibited by the selection agent (ammonium glufosinate), and which are usually and mainly made up of cells resulting from the division of a cell having integrated into its genetic inheritance one or more copies of the selection gene. The transformation efficiency is 10%.

These calluses are identified, individualized, amplified and then cultivated so as to regenerate plantlets. In order to avoid any interference with nontransformed cells, all these operations are carried out on culture media containing the selective agent.

Acclimatization:

The acclimatization of the plantlets is carried out when the latter have developed sufficiently on T1+G2, i.e. when the roots reach the bottom of the tube and when the ridged axis is sufficiently rigid and developed. The plantlets are acclimatized in a phytotron in small pots with slightly enriched compost. The small pots are arranged on a bench located 1.5 meters from the lamps. To maximize the obtaining of progeny, the acclimatization of 2 plantlets in a phytotron is necessary. On average, two weeks are necessary for weaning the plantlets.

The plants thus regenerated and acclimatized are then cultivated in a greenhouse where they can be crossed or self-fertilized.

This embodiment consisting in producing a fertility-restoring maize line heterozygous for the fertility-restoring gene (SSB/+) by cotransformation of a maize plant with the plasmids of Examples 2a, 2b and 3a is the first embodiment.

A second embodiment consisting in producing a fertility-restoring maize line heterozygous for the fertility-restoring gene (SSB/+) consists in cotransforming a maize plant with the plasmids of Examples 2a and 3a according to the same protocol as described above.

A third embodiment consisting in producing a fertility-restoring maize line heterozygous for the fertility-restoring gene (SSB/+) consists in cotransforming a maize plant with the plasmids of Examples 2b and 3a according to the same protocol as described above.

5.b) Production of a Fertility-Restoring Maize Line Heterozygous for the Fertility-Restoring Gene (SSB/+) by Cotransformation of Maize with the Plasmids of Examples 2c (Restorer Plasmid pRec 4962) and 3b (Selection Plasmid pRec 273)

The production of the fertility-restoring maize line according to the present Example 5b is carried out by cotransformation of a maize plant with the plasmids described according to Examples 2c and 3b according to the Agrobacterium tumefaciens transformation technique known to those skilled in the art.

The bar gene (gene encoding the selection agent) is eliminated during the cotransformation. 10% of the transformants obtained contain the 2 expression cassettes contained in the plasmids of Examples 2c and 3b. There will be segregation in the progeny.

This embodiment (4^{th} embodiment) consisting in producing a fertility-restoring maize line heterozygous for the fertility-restoring gene (SSB/+) is the preferred embodiment according to this present invention. However, other transformation techniques known to those skilled in the art may be used.

5.c) Molecular Characterization of the Transformants

The transformant SSB-001a obtained according to Example 5a) was characterized by the same methodology as that described according to Example 4c of the present invention. The molecular analysis was carried out on the transformant SSB-001a resistant to the herbicide Basta® (plant No. 12928), on a sister plant resistant to the herbicide Basta® (plant No. 12929) and on 2 plants sensitive to the herbicide (13008 and 13061). These 4 plants are derived from the ear of the primary transformant SSB-001a. The genomic DNA (14 μg) of the plants was digested with the Eco RV and Hind III restriction enzymes. Three probes were used for the analysis: A9 promoter (pA9), HMWG promoter (pHMWG) and. Bar.

The results of the molecular hybridizations with the Bar probe indicate that the molecular profiles are identical for the two plants resistant to the herbicide Basta®. The hybridizations with the pA9 and pHMWG probes reveal a large number of bands, reflecting the integration of several copies of the plasmids p3222 and p3223. The hybridization with the Bar probe shows a simple profile, a major band is revealed for the 2 enzymes Eco RV and Hind III. The size of the fragment revealed by the Hind III digestion is 8 kb. The bands of very weak intensity correspond to the very intense signals revealed previously with the pA9 and pHMWG probes.

### Example 6

### Crossing of the Heterozygous Male Sterile Maize Line (AMS/+) with the Fertility-Restoring Maize Line Heterozygous for the Fertility-Restoring Gene (SSB/+): Obtaining F1 Plants

The heterozygous male sterile maize line (AMS/+) resistant to the herbicide Basta® obtained according to Example 4a is crossed with the fertility-restoring maize line heterozygous for the fertility-restoring gene (SSB/+), plant resistant to the herbicide Basta®, obtained according to Example 5 in order to obtain the F1 plants. The same type of cross can be carried out between the heterozygous male sterile maize line (AMS/+) obtained according to Example 4b and the fertility-restoring maize line heterozygous for the fertility-restoring gene (SSB/+), obtained according to Example 5. The fertilization is carried out manually by means of a technique known to those skilled in the art. The male sterile plant is brought to flowering, and the pollen of the (SSB/+) plant is deposited onto the bristles of the male sterile line.

At the end of this cross, genetic analyses are carried out on the F1 plants. The genetic analyses consist of counting, in relation to the presence of the various markers, among the progeny.

All the theoretical frequencies mentioned are true if, and only if, there is no linkage between the AMS transgene and the fertility-restoring gene.

Genetic Assessment of the F1:

| | AMS | + |
|---|---|---|
| SSB | SSB/+; AMS/+ | SSB/+; +/+ |
| + | AMS/+; +/+ | +/+; +/+ |

Phenotypic Assessment of the F1:

| | | |
|---|---|---|
| Seed | Seed | Basta segregation |

| phenotype | segregation | Resistant | Sensitive |
|---|---|---|---|
| Normal | 50% | 50% | 50% |
| Deficient | 50% | 100% | 0% |

Half the F1 is therefore made up of normal seeds, the other half being deficient seeds (“small seed” phenotype caused by the shrunken2 and brittle2 genes in the antisense orientation).

For each F1, we evaluated the segregation for the glufosinate resistance in order to detect the batches of deficient seeds.

The deficient seeds are selected by visual separation with respect to the maize ear. These seeds are all resistant to the herbicide Basta®.

The deficient seeds, which have the genotype (AMS/+; SSB/+) or (+/+; SSB/+), are selected and then sown and germinated.

### Example 7

### Self-Fertilization of the F1 Plants for Production of the F2

The F1 plants resistant to the herbicide Basta® derived from deficient seeds according to Example 6 are self-fertilized in order to obtain the F2 plants.

Since the F1 plants derived from deficient seeds consist of both plants of genotype (SSB/+; +/+) and plants of genotype (SSB/+; AMS/+), there are then two cases of self-fertilization:

7.a) Self-Fertilizations of the Plants of Genotype (SSB/+; +/+)

The F1 plants of genotype (SSB/+; +/+) obtained according to Example 6 are self-fertilized. At the end of this self-fertilization, the genetic analyses are carried out on the F2 plants:

Genetic Assessment of the F2:

| | SSB | + |
|---|---|---|
| SSB | SSB/SSB; +/+ | SSB/+; +/+ |
| + | SSB/+; +/+ | +/+; +/+ |

Phenotypic Assessment of the F2:

| | | |
|---|---|---|
| Seed | Seed | Basta segregation |

| phenotype | segregation | Resistant | Sensitive |
|---|---|---|---|
| Normal | 25% | 0% | 100% |
| Deficient | 75% | 100% | 0% |

This step comprising self-fertilizations of the plants of genotype (SSB/+; +/+) can advantageously be eliminated by means of a step consisting in genotyping by carrying out a PCR specific for the AMS transgene on the F1 plants derived from deficient seeds according to Example 6. The deficient seeds are germinated, the young maize plants are self-fertilized, and molecular separation is carried out by PCR. Only the plants positive for detection of the AMS transgene by PCR are conserved.

The primers specific for the AMS gene of interest (pA9-barnase-CaMV3′) which allows its amplification by PCR are as follows:

| | | |
|---|---|---|
| Name | Orientation | Sequence |

| | | | |
|---|---|---|---|
| A9A | Direct | TAGACATTGTAGGTTGGTTTTG | (SEQ ID No. 9) |
| (Pro A9) | | | |
| Barn 1 | Direct | GCACAGGTTATCAACACGTTTGAC | (SEQ ID No. 10) |
| (barnase) | | | |
| Barn 4 | Direct | ATCCGGCCATTTCTGAAGAGAA | (SEQ ID No. 11) |
| (barnase) | | | |
| A9B | Reverse | TCTAGTTACTTCATAAGTTTTG | (SEQ ID No. 12) |
| (Pro A9) | | | |
| Barn 6 | Reverse | TTGCGGGTTTGTGTTTCCATATTG | (SEQ ID No. 13) |
| (barnase) | | | |
| CaMVol1 | Reverse | ATTGATAAGGGGTTATTAGGGG | (SEQ ID No. 14) |
| (3′ CaMV) | | | |

The size of the fragment amplified using the pairs of primers A9A/A9B, Barn1/Barn6 or Barn4/CaMVoll is 799 bp, 880 bp or 979 bp, respectively.

7.b) Self-Fertilizations of the Plants of Genotype (AMS/+; SSB/+)

The F1 plants of genotype (AMS/+; SSB/+) obtained according to Example 6 are self-fertilized in order to obtain the F2 progeny. At the end of this self-fertilization, the genetic analyses are carried out on the F2 progeny:

Genetic Assessment of the F2:

| | AMS; + | +; SSB | AMS; SSB | +; + |
|---|---|---|---|---|
| AMS; + | AMS/AMS; | AMS/+; | AMS/AMS; | AMS/+; |
| | +/+ | SSB/+ | SSB/+ | +/+ |
| +; SSB | AMS/+; SSB/+ | SSB/SSB; | AMS/+; SSB/SSB | SSB/+; +/+ |
| | | +/+ | | |
| AMS; | AMS/AMS; | AMS/+; | AMS/AMS; | AMS/+; |
| SSB | SSB/+ | SSB/SSB | SSB/SSB | SSB/+ |
| +; + | AMS/+; +/+ | SSB/+; +/+ | AMS/+; SSB/+ | +/+; +/+ |

Phenotypic Assessment of the F2:

| | | |
|---|---|---|
| Seed | Seed | Basta segregation |

| phenotype | segregation | Resistant | Sensitive |
|---|---|---|---|
| Normal | 25% | 75% | 25% |
| Deficient | 75% | 100% | 0% |

The genetic analysis of the F2 progeny on the seeds that are normal (25%) for the expression of the barnase gene with resistance to the herbicide Basta® makes it possible to determine the F1 hybrid plants which exhibited the genotype (SSB/+; AMS/+). In terms of the F2 plants derived from the deficient seeds, ⅙ have the genotype (AMS/AMS; SSB/+). As regards the F2 plants which have normal seeds, ¼ have the genotype (AMS/AMS; +/+).

7.c) Results

14 F2 ears were produced. For each of these ears, separation was carried out with respect to seed genotype (deficient or not). 14 batches of transgenic seeds (from A to N) were therefore analyzed. These 14 seed batches (batch A to batch N) were divided in two as a function of their normal or deficient phenotype. The code used for the 28 sets of seeds thus prepared is, for example, for batch A: A01=normal seeds, A02=“deficient” seeds.

### Example 8

### Sowing of the Deficient Seeds of the F2 Progeny and Genotyping of the Plants (AMS/AMS; SSB/+)

Directed sowing of the F2 seeds obtained above, as a function of the deficient or normal seed phenotype, and then detection of the (AMS/AMS; SSB/+) plants by genotyping were carried out.

8.a) Sowing of the F2 Progeny

Directed sowing of the 14 families was carried out and, at the 3- to 4-leaf stage, a 0.5% Basta spray test was carried out. The results of this test are described in the table below:

| | | | Number of | | Number of | | Number of | |
|---|---|---|---|---|---|---|---|---|
| | | Number of | germinations | | resistants | | sensitives | |
| | Seed | seeds | / seeds | | / seeds | | / seeds | |
| Code | phenotype | sown | sown | % | sown | % | sown | % |
| A 01 | normal | 30 | 30 | 100 | 27 | 90 | 3 | 10 |
| A 02 | deficient | 60 | 60 | 100 | 60 | 100 | 0 | 0 |
| B 01 | normal | 30 | 28 | 93.3 | 0 | 0 | 24 | 85.7 |
| B 02 | deficient | 60 | 60 | 100 | 60 | 100 | 0 | 0 |
| C 01 | normal | 30 | 30 | 100 | 19 | 63.3 | 11 | 36.7 |
| C 02 | deficient | 60 | 59 | 98.3 | 56 | 94.9 | 3 | 5.1 |
| D 01 | normal | 30 | 30 | 100 | 20 | 66.7 | 10 | 33.3 |
| D 02 | deficient | 60 | 60 | 100 | 57 | 95 | 3 | 5 |
| E 01 | normal | 30 | 30 | 100 | 19 | 63.3 | 11 | 36.7 |
| E 02 | deficient | 50 | 48 | 96 | 48 | 100 | 0 | 0 |
| F 01 | normal | 30 | 30 | 100 | 2 | 6.7 | 28 | 93.3 |
| F 02 | deficient | 60 | 60 | 100 | 60 | 100 | 0 | 0 |
| G 01 | normal | 30 | 29 | 96.7 | 21 | 72.4 | 8 | 27.6 |
| G 02 | deficient | 60 | 59 | 98.3 | 59 | 100 | 0 | 0 |
| H 01 | normal | 15 | 15 | 100 | 7 | 46.7 | 8 | 53.3 |
| H 02 | deficient | 30 | 29 | 96.7 | 29 | 100 | 0 | 0 |
| I 01 | normal | 30 | 30 | 100 | 0 | 0 | 24 | 80 |
| I 02 | deficient | 60 | 60 | 100 | 60 | 100 | 0 | 0 |
| J 01 | normal | 30 | 30 | 100 | 0 | 0 | 27 | 90 |
| J 02 | deficient | 60 | 59 | 98.3 | 55 | 93.2 | 4 | 6.8 |
| K 01 | normal | 30 | 30 | 100 | 21 | 70 | 9 | 30 |
| K 02 | deficient | 60 | 60 | 100 | 58 | 96.7 | 5 | 8.3 |
| L 01 | normal | 30 | 30 | 100 | 0 | 0 | 29 | 96.7 |
| L 02 | deficient | 60 | 60 | 100 | 60 | 100 | 0 | 0 |
| M 01 | normal | 30 | 30 | 100 | 22 | 73.3 | 7 | 23.3 |
| M 02 | deficient | 60 | 60 | 100 | 60 | 100 | 0 | 0 |
| N 01 | normal | 30 | 28 | 93.3 | 20 | 71.4 | 7 | 25 |
| N 02 | deficient | 60 | 60 | 100 | 60 | 100 | 0 | 0 |

The results of the test made it possible to eliminate the families B, F, I, J and L which resulted from the self-fertilization of F1 plants with the genotype (SSB/+; +/+).

The families A, C, D, E, G, H, K, M and N were conserved. These ears resulted from the self-fertilization of F1 plants with the genotype (SSB/+; AMS/+). Details of the genetic and phenotypic assessments of the self-fertilization of these plants were given in Example 7b. In these selected families, all the plants resistant to the herbicide Basta® derived from normal seeds (batches ending with 01) and up to 40 plants resistant to the herbicide Basta® derived from deficient seeds were kept for cultivation.

8.b) Identification of the Plant Genotype (AMS/AMS; SSB/+) by Genotyping and Southern Blotting

A molecular analysis is carried out in order to identify the plants homozygous to the transgene derived from STB-27b (AMS/AMS) and heterozygous for the transgene derived from SSB001a (SSB/+).

The genomic DNA of the progeny was extracted from 50 mg of leaves according to the protocol and use of the extraction kit: Qiagen Dneasy 96 plant kit (Qiagen SA, 91974 Courtaboeuf cedex, France). Southern methodology is used to identify the molecular profiles.

For each individual, the presence of the two Bar genes: that coming from STB27b and that coming from SSB001a, is visualized. For this, the DNAs were digested with the Hind III enzyme and then hybridized with the Bar probe. According to the molecular analyses carried out on the parent transformants, it is known that a fragment 8 kb in size corresponds to the copy of the Bar gene derived from the transformant SSB001a and a fragment 1.7 kb in size corresponds to the copy of the Bar gene derived from the transformant STB27b. For a given plant, the intensity of the hybridization signals is also evaluated, in order to identify the zygosity (heterozygosity or homozygosity) of the plants for the Bar gene under consideration (SSB001a or STB27b). This analysis therefore allows us to identify the genotype of the plants derived from the SSB×STB cross. According to the prior selection of the seeds from which the plants are derived, 6 genotypes are expected and presented in Table 1. Table 1:

Genotype

| Number of copies of | Number of copies of | | |
|---|---|---|---|
| Bar from SSB-001a | Bar from STB-27b | | theoretical % |
| 2 | 0 | A | 8.3 |
| 2 | 1 | B | 16.7 |
| 2 | 2 | C | 8.3 |
| 1 | 0 | D | 16.7 |
| 1 | 1 | E | 33.3 |
| 1 | 2 | F | 16.7 |
| Total | | | 100 |

The genotype sought in this study is homozygosity for the STB-27b T-DNA (“2 copies” of the Bar gene) and heterozygosity for the SSB-001a Bar gene (“1 copy” of the Bar gene).

Southern blots were performed with the DNA from 8 daughter plants derived from an ear, on the 9 ears selected. 58 plants were genotyped according to the protocol described above. All the expected genotypes are represented among the 9 plants analyzed (2 different ears).

In total, 10 plants exhibit the genotype of interest, namely: homozygosity for the STB-27b T-DNA (“2 copies” of the Bar gene) and heterozygosity for the SSB-001a Bar gene (“1 copy” of the Bar gene). The frequencies observed are very close to the expected theoretical frequencies whatever the genotype under consideration (Table 2).

TABLE 2

| Genotype | | | | |
|---|---|---|---|---|
| Number of | Number of | Number | | |
| copies of Bar | copies of Bar | of | | |
| from SSB-001a | from STB-27b | plants | observed % | theoretical % |
| 2 | 0 | 3 | 5.2 | 8.3 |
| 2 | 1 | 9 | 15.5 | 16.7 |
| 2 | 2 | 3 | 5.2 | 8.3 |
| 1 | 0 | 11 | 19 | 16.7 |
| 1 | 1 | 22 | 37.9 | 33.3 |
| 1 | 2 | 10 | 17.2 | 16.7 |
| Total | | 58 | 100 | 100 |

### Example 9

### Obtaining Prebase Seeds (AMS/AMS; +/+), Base Seeds (AMS/+; +/+) and Hybrid Seeds

Many crosses can be envisioned in order to produce prebase seeds, base seeds and hybrid seeds. The crosses described below are are not limiting:

9.a) Self-Fertilization of the Plants (AMS/AMS; SSB/+) in Order to Obtain Prebase Seeds

The advantage of this cross is to multiply the line with the genotype (AMS/AMS; SSB/+) and also to produce seeds with the genotype (AMS/AMS; +/+) or prebase seeds.

In fact, self-fertilizations produce seed homozygous for the transgene conferring male sterility (at the F3 level, all the normal seeds will have the genotype (AMS/AMS; +/+)). At the end of this self-fertilization, the genetic analyses are carried out:

Genetic Assessment:

| | AMS; SSB | AMS; + | AMS; SSB | AMS; + |
|---|---|---|---|---|
| AMS; | AMS/AMS; | AMS/AMS; | AMS/AMS; | AMS/AMS; |
| SSB | SSB/SSB | SSB/+ | SSB/SSB | SSB/+ |
| AMS; + | AMS/AMS; SSB/+ | AMS/AMS; | AMS/AMS; | AMS/AMS; |
| | | +/+ | SSB/+ | +/+ |
| AMS; | AMS/AMS; | AMS/AMS; | AMS/AMS; | AMS/AMS; |
| SSB | SSB/SSB | SSB/+ | SSB/SSB | SSB/+ |
| AMS; + | AMS/AMS; SSB/+ | AMS/AMS; | AMS/AMS; | AMS/AMS; |
| | | +/+ | SSB/+ | +/+ |

Phenotypic Assessment:

| | | |
|---|---|---|
| Seed | Seed | Basta segregation |

| phenotype | segregation | Resistant | Sensitive |
|---|---|---|---|
| Normal | 25% | 100% | 0% |
| Deficient | 75% | 100% | 0% |

All of the normal seeds have the genotype (AMS/AMS; +/+). These are the prebase seeds. 75% of the deficient seeds have these genotype (AMS/AMS; SSB/+). The plants derived from these deficient seeds can be self-fertilized in order to multiply (maintain) the plants of genotype (AMS/AMS; SSB/+). The young plant of genotype (AMS/AMS; SSB/+) is also called sterility-maintaining young plant.

9.b) Crossing of the Plants of Genotype (AMS/AMS; SSB/+) with the Elite Line Having a Wild-Type Genotype (WT) in Order to Obtain Seeds of Genotype (AMS/+; +/+)

The plants derived from deficient seeds are crossed with a WT elite line. At the end of this cross, the genetic analyses are carried out:

Genetic Assessment:

| | AMS; SSB | AMS; + | AMS; SSB | AMS; + |
|---|---|---|---|---|
| +; + | AMS/+; SSB/+ | AMS/+; +/+ | AMS/+; SSB/+ | AMS/+; +/+ |

Phenotypic Assessment:

| | | |
|---|---|---|
| Seed | Seed | Basta segregation |

| phenotype | segregation | Resistant | Sensitive |
|---|---|---|---|
| Normal | 50% | 100% | 0% |
| Deficient | 50% | 100% | 0% |

Two scenarios occur:
in ⅔ of the cases, the F2 plants had the genotype (AMS/+; +/+). On these F3 ears, only normal seeds with a 50/50 segregation with respect to the herbicide Basta® are obtained. These ears are of no interest to us;
in ⅓ of the cases, the F2 plants had the genotype (AMS/AMS; +/+). On these F3 ears, only normal seeds with a genotype (AMS/+; +/+) 100% resistant to the herbicide Basta® are obtained. These are the ears which interest us. The normal seeds of genotype (AMS/+; +/+) constitute the base seeds.

20 seeds from each F2 ear are sown and the ears of interest are identified as a function of resistance to the herbicide Basta®.

9.c) Crossing of the Plants of Genotype (AMS/AMS; +/+) with the Elite Line Having a Wild-Type Genotype (WT) in Order to Obtain Seeds of Genotype (AMS/+; +/+)

The plants derived from the seeds of genotype (AMS/AMS; +/+) are crossed with an elite line. Preferably, this elite line is identical to that used in the step of successive backcrosses in order to introgress the genotype (AMS/AMS; SSB/+).

The interest of this cross is to produce the base seed of genotype (AMS/+; +/+).

Genetic Assessment:

| | AMS | AMS |
|---|---|---|
| + | AMS/+; +/+ | AMS/+; +/+ |
| + | AMS/+; +/+ | AMS/+; +/+ |

Phenotypic Assessment

| | | |
|---|---|---|
| Seed | Seed | Basta segregation |

| phenotype | segregation | Resistant | Sensitive |
|---|---|---|---|
| Normal | 100% | 100% | 0% |

All the seeds (which are normal) have the genotype (AMS/+; +/+) and, consequently, will give male sterile plants.

9.d) Crossing of the Plants of Genotype (AMS/+; +/+) with the Male Elite Line (WT) in Order to Obtain Hybrid Seeds

Plants derived from the seeds of genotype (AMS/+; +/+) are crossed with a male elite line. Preferably, the elite line used in this cross is different from that used in the cross described in Example 9.c).

The interest of this cross is to produce the hybrid seed.

Genetic Assessment:

| | AMS | + |
|---|---|---|
| + | AMS/+; +/+ | +/+; +/+ |
| + | AMS/+; +/+ | +/+; +/+ |

Phenotypic Assessment:

| | | |
|---|---|---|
| Seed | Seed | Basta segregation |

| phenotype | segregation | Resistant | Sensitive |
|---|---|---|---|
| Normal | 100% | 50% | 50% |

All the seeds are normal. Half of them will give male sterile plants, the other half will give male fertile plants, thus ensuring pollination and, consequently, the production of maize “for consumption” or “seed” maize.

### Example 10

### Separation of the Deficient Seeds by Means of a Densimetric Table

The densimetric table used makes it possible to divide up into six fractions seeds of equivalent size and superficial quality, but which differ from one another by virtue of their specific weight.

Densimetric separation is carried out on a batch of seeds from F3 ears, derived from the self-fertilization of plants of genotype (+/+; SSB/+). Theoretically, these ears comprise 75% of deficient seeds and 25% of normal seeds. 33 ears were shelled so as to obtain approximately 500 grams of seed. 6 seed fractions were constituted by densimetric separation.

The table below is a summary of the results obtained (separation with respect to the phenotype of the seed and genetic analyses regarding the expression of the bar gene conferring glufosinate resistance).

| | | Seed phenotype | | Genetic analysis results | | | | | | |
|---|---|---|---|---|---|---|---|---|---|---|
| | Total | seperation | | | Number | | Number | | Number | |
| | number | Number of | Number of | Number of | germinations | | resistants | | sensitives | |
| | of seeds | normal seeds | deficient seeds | seeds sown | /seeds sown | % | /germinations | % | /germinations | % |
| Fraction 1 | 50 | 0 | 50 | 40 | 36 | 90 | 36 | 100 | 0 | 0 |
| Fraction 2 | 69 | 0 | 69 | 60 | 49 | 81.7 | 49 | 100 | 0 | 0 |
| Fraction 3 | 445 | 27 | 418 | 160 | 156 | 97.5 | 149 | 96 | 7 | 4 |
| Fraction 4 | 97 | 8 | 89 | 80 | 80 | 100 | 72 | 90 | 8 | 10 |
| Fraction 5 | 540 | 103 | 437 | 160 | 154 | 96.3 | 120 | 78 | 34 | 22 |
| Fraction 6 | 779 | 756 | 23 | 160 | 155 | 96.9 | 6 | 4 | 149 | 96 |

Separation with Respect to the Seed Phenotype (Normal or Deficient):

Fractions 1 and 2 comprise only deficient seeds.

In fractions 3, 4 and 5, the seeds are essentially deficient seeds, but some normal seeds are also found (i.e. 6.1, 8.2 and 19.1%, respectively, for fractions 3, 4 and 5).

As regards fraction 6, there are virtually only normal seeds (97%).

Fractions 1 to 5 therefore correspond to the deficient seeds and fraction 6 to the normal seeds.

A small quantity of nonseparated seeds is also found.

This small quantity corresponds both to normal seeds and to deficient seeds. However, visually, there is a greater number of deficient seeds compared to the normal seeds.

Genetic Analysis Results (Regarding Expression of the Bar Gene Conferring Resistance to the Herbicide Basta®):

The genetic analysis results reinforce those of the separation carried out with regard to the seed phenotype for each of the fractions.

In fraction 6, there is therefore a small portion of deficient seeds (approximately 3 to 4%). The latter is completely removed through being passed over the densimetric table a second time.

Fractions 1 to 5 correspond to the deficient seeds and fraction 6 to the normal seeds.

### Example 11

### Evaluation of the Various Steps of the Production of Hybrid Maize Seeds Using this Novel Method

Three types of experiment were tested for seed production:
Production of prebase seeds
Production of base seeds
Product of hybrid seeds
   Production of Prebase Seeds:

The production of prebase seeds (genotype (AMS/AMS; +/+)) was provided by the cultivation of plants of genotype (AMS/AMS; SSB/+). These plants are indeed observed to be male fertile.

The plants of genotype (AMS/AMS; SSB/+) are self-fertilized. Approximately 25% of seeds obtained in the progeny correspond to prebase seeds.

Production of Base Seeds:

The production of base seeds (genotype (AMS/+; +/+)) was provided by the cultivation of plants of genotype (AMS/AMS; +/+). These plants are indeed observed to be male sterile and exhibit no deleterious effect on the vegetative aspect of the plant, demonstrating that the use of an AMS transgene in the homozygous state is compatible with the system for producing hybrid maize seeds as described in the present invention.

The young plants of genotype (AMS/AMS; +/+) were crossed with a young plant having a wild-type genotype. 100% of the seeds obtained in the progeny correspond to base seeds.

Production of Hybrid Seeds:

The production of hybrid seeds was obtained by crossing between male sterile plants of genotype (AMS/+; +/+) derived from the base seeds, with a wild-type elite line. All the young plants of genotype (AMS/+; +/+) indeed showed complete male sterility (100% sterility).

### Example 12

### Production of Hybrid Seeds

Two types of crosses are preferably carried out in seed production. These two crosses make it possible to produce hybrid seed.

12.a) Crossing of the Young Plants of Genotype (AMS/AMS; SSB/+) with Young Plants of Genotype (AMS/AMS; +/+)

Genetic Assessment:

| | AMS; SSB | AMS; + | AMS; SSB | AMS; + |
|---|---|---|---|---|
| AMS; + | AMS/AMS; SSB/+ | AMS/AMS; | AMS/AMS; | AMS/AMS; |
| | | +/+ | SSB/+ | +/+ |
| AMS; + | AMS/AMS; SSB/+ | AMS/AMS; | AMS/AMS; | AMS/AMS; |
| | | +/+ | SSB/+ | +/+ |

Phenotypic Assessment:

| | | |
|---|---|---|
| Seed | Seed | Basta segregation |

| phenotype | segregation | Resistant | Sensitive |
|---|---|---|---|
| Normal | 50% | 100% | 0% |
| Deficient | 50% | 100% | 0% |

50% of the seeds obtained are normal and all have the genotype (AMS/AMS; +/+). These seeds can be separated by densimetric separation, preferably using a densimetric table.

12.b) Crossing of the Plants of Genotype (AMS/AMS; SSB/+) with an Elite Line Having a Wild-Type Genotype (WT)

Genetic Assessment:

| | AMS; SSB | AMS; + | AMS; SSB | AMS; + |
|---|---|---|---|---|
| +; + | AMS/+; SSB/+ | AMS/+; +/+ | AMS/+; SSB/+ | AMS/+; +/+ |

Phenotypic Assessment:

| | | |
|---|---|---|
| Seed | Seed | Basta segregation |

| phenotype | segregation | Resistant | Sensitive |
|---|---|---|---|
| Normal | 50% | 100% | 0% |
| Deficient | 50% | 100% | 0% |

All the normal seeds have the genotype (AMS/+; +/+). These seeds can be separated by densimetric separation, preferably using a densimetric table.

This cross makes it possible to gain time in obtaining the hybrid seeds (6 to 12 months less time) compared with the cross described in Example 12.a). On the other hand, it requires a surface for production of the maintaining line (of genotype AMS/AMS; SSB/+) which is greater in size for an equivalent number of (hybrid) seed hectares.

### BIBLIOGRAPHIC REFERENCES

An et al. (1986) Plant Physiol. 81, 86-91.
Anderson, O. D., Green, F. C., Yip, R. E., Halford, N. G., Shewry, P. R. and Malpica-Romero, J. M. (1989) Nucl. Acid. Res. 17, 461.
Bevan et al. (1984) Nucleic Acid Research, 11, 369-385.
Cao J., Duan, X., McElroy, D. and Wu, R. (1992) Plant. Cell. Rep. 11, 586-591.
Cheng W. H., Taliercio E. W. and Chourey P. S. (1996). The Miniaturel Seed Locus of Maize Encodes a Cell Wall Invertase Required for Normal Development of Endosperm and Maternal Cells in the Pedicel. Plant Cell., 8(6):971-983.
Chourey, P. S., Nelson, O. E. (1976). The enzymatic deficiency conditioned by the shrunken-1 mutations in maize. Biochem Genet, 14(11-12):1041-55.
Depicker et al., (1982) J. Mol. Appl. Genet., 1, 561-573.
Di Fonzo N, et al. (1988) Mol Gen Genet 212(3), 481-7.
Finer J. (1992) Plant Cell Report 11: 323-328.
Franck et al. (1980) Cell, 21, 285-294.
Fromm et al. (1986) Nature, 319, 791-793.
Hartley, R.W (1988) J. Mol. Biol. 202, 913-915.
Herrera-Estrella et al. (1983) EMBO J. 2, 987-995.
Ishida Y, Saito H, Ohta S, Hiei Y, Komari T, Kumashiro T (1996) Nat. Biotechnol. 14(6):745-50.
Jouanin et al. (1987) Plant Sci., 53, 53-63.
Klein (1987) Nature 327:70-73.
Komari T., Hiei Y., Saito Y., Murai N., Kumashiro T. (1996). Vectors carrying two separate T-DANs for co-transformation of higher plants mediated by Agrobacterium tumefaciens and segregation of transformants free from selection markers. Plant Journal, 10, 165-174.
Krens et al. (1982) Nature, 296, 72-74.
Lyzrik L. and Hodges T. (1997). FLP mediated recombination of FRT sites in the maize genome. Nucleic Acids Research, 44, 123-132.
McElroy D., Blowers, A. D., Jenes, B. and Wu, R. (1991) Mol. Gen. Genet. 231, 150-160.
Messing J. et al. (1983) Methods in Enzymology, 101, 20-78.
Paul, W., et al. (1992) Plant Mol. Biol. 19: 611-622.
Robert et al. (1989) Plant Cell, 1: 569-578.
Sambrook, Fritsch & Maniatis, Molecular Cloning: A Laboratory Manual, Second Edition (1989) Cold Spring Harbor Laboratory Press, Cold Spring Harbor, New York.
Southern E. M. (1975) J. Mol Biol. 98: 503-517.
Watson et al. (1994) Ed. De Boeck University, pp. 273-292.
White, J., Chang S-YP., Bibb, M. J. and Bibb, M. J. (1990) Nucl. Acid Res. 18, 1062.
Yoder et al. (1993) Bio/Technology, 12, 263-292.

1. A method for the production of maize seeds homozygous for a transgene conferring artificial nuclear male sterility (“AMS”) and heterozygous for a fertility-restoring gene linked to a “small seed” phenotype marker, comprising the steps consisting in:
a) crossing a male sterile maize plant heterozygous for the AMS transgene with a fertility-restoring maize plant comprising in its genome a fertility-restoring gene linked to a “small seed” phenotype marker, b) selecting, by means of the “small seed” phenotype, the maize seeds comprising in their genome a fertility-restoring gene linked to a “small seed” phenotype marker, c) self-fertilizing the maize plants derived from seeds selected according to step b), d) selecting the seeds homozygous for the AMS transgene and heterozygous for the fertility-restoring gene linked to a “small seed” phenotype marker.

2. A method for the production of maize seeds homozygous for a transgene conferring artificial nuclear male sterility (“AMS”) and heterozygous for a fertility-restoring gene linked to a “small seed” phenotype marker, comprising the steps consisting in:
a) crossing a male sterile maize plant heterozygous for the AMS transgene with a fertility-restoring maize plant comprising in its genome a fertility-restoring gene linked to a “small seed” phenotype marker,
b) genotyping the seeds obtained by means of the cross according to step a),
c) self-fertilizing the maize plants derived from the seeds genotyped according to step b),
d) selecting the seeds homozygous for the AMS transgene and heterozygous for the fertility-restoring gene linked to a “small seed” phenotype marker.

3. A maize seed homozygous for an AMS transgene and heterozygous for a fertility-restoring gene linked to a “small seed” phenotype marker, which can be obtained by the method as claimed in claim 1.

4. A method for the production of maize seeds homozygous for a transgene conferring artificial nuclear male sterility (“AMS”), comprising the steps consisting in:
a) crossing a male sterile maize plant heterozygous for the AMS transgene with a fertility-restoring maize plant comprising in its genome a fertility-restoring gene linked to a “small seed” phenotype marker,
b) selecting, by means of the “small seed” phenotype, the maize seeds comprising in their genome a fertility-restoring gene linked to a “small seed” phenotype marker,
c) self-fertilizing the maize plants derived from the seeds selected according to step b),
d) selecting seeds homozygous for the AMS transgene and heterozygous for the fertility-restoring gene linked to a “small seed” phenotype marker,
e) self-fertilizing maize plants derived from seeds according to step d),
f) selecting seeds homozygous for the AMS transgene.

5. A method for the production of maize seeds homozygous for a transgene conferring artificial nuclear male sterility (“AMS”), comprising the steps consisting in:
a) crossing a male sterile maize plant heterozygous for the AMS transgene with a fertility-restoring maize plant comprising in its genome a fertility-restoring gene linked to a “small seed” phenotype marker,
b) genotyping the seeds obtained by means of the cross according to step a),
c) self-fertilizing the maize plants derived from the seeds genotyped according to step b),
d) selecting the seeds homozygous for the AMS transgene and heterozygous for the fertility-restoring gene linked to a “small seed” phenotype marker,
e) self-fertilizing maize plants derived from seeds according to step d),
f) selecting seeds homozygous for the AMS transgene.

6. A method for the production of maize seeds homozygous for an AMS transgene, comprising the steps consisting in:
a) self-fertilizing maize plants derived from seeds as claimed in claim 3,
b) selecting seeds homozygous for an AMS transgene.

7. The method as claimed in claim 1, characterized in that at least one selection step comprises densimetric separation.

8. The method as claimed in claim 7, characterized in that the densimetric separation is carried out using a densimetric table.

9. A method for the production of a seed heterozygous for an AMS transgene, comprising the crossing of a maize plant derived from a seed homozygous for an AMS transgene, which can be obtained by the method as claimed in claim 4, with a maize plant having a wild-type genotype.

10. A method for the production of a seed heterozygous for an AMS transgene, characterized in that the method as claimed in claim 4 also comprises the crossing of a maize plant derived from said seed homozygous for an AMS transgene, with a maize plant having a wild-type genotype.

11. The method as claimed in claim 1, in which the AMS transgene conferring artificial nuclear male sterility is the barnase gene, which is included in an expression cassette, under the control of a promoter specific for pollen formation, in particular an anther-specific promoter such as pA3, pA6, pA9, pTA29, or of the Mac2 promoter, and of the CaMV 3′ or Nos 3′ terminator, genetically linked to a gene encoding a selection agent under the control of the actin promoter-actin intron and of the CaMV 3′ or Nos 3′ terminator.

12. The method as claimed in claim 11, characterized in that the expression cassette comprising the barnase gene also comprises a gene encoding a protein of therapeutic and/or prophylactic interest genetically linked to the barnase gene.

13. The method as claimed in claim 11, characterized in that said promoter is the pA9 promoter specific for pollen formation.

14. The method as claimed in claim 11, characterized in that said gene encoding a selection agent is chosen from the bar gene which confers resistance to the herbicide Basta® and the NptII gene which confers resistance to kanamycin, said gene being included within the Ds transposable element.

15. An expression cassette comprising a fertility-restoring gene genetically linked to at least one gene encoding a “small seed” phenotype, combined with elements which allow their expression in plant cells, in particular a transcription promoter and terminator.

16. The expression cassette as claimed in claim 15, characterized in that said fertility-restoring gene is the barstar gene placed under the control of a promoter specific for pollen formation, in particular an anther-specific promoter such as pA3, pA6, pA9, pTA29, or of the Mac2 promoter, and of the CaMV 3′ or Nos 3′ terminator, genetically linked to a gene encoding a selection agent under the control of the actin promoter-actin intron and of the CaMV 3′ or Nos 3′ terminator.

17. The expression cassette as claimed in claim 15, characterized in that said gene encoding a “small seed” phenotype is chosen from the shrunken 2 and brittle 2 genes in antisense orientation.

18. The expression cassette as claimed in claim 15, characterized in that the promoter combined with the gene encoding a “small seed” phenotype is chosen from the HMWG and B32 promoters.

19. The expression cassette as claimed in claim 15, characterized in that said terminator is chosen from the Nos 3′ terminator and the CaMV 3′ terminator.

20. A vector, in particular a plasmid, characterized in that it contains at least one expression cassette as described in claim 11.

21. A cellular host, in particular a bacterium such as Agrobacterium tumefaciens transformed with a vector as claimed in claim 20.

22. A maize cell transformed with at least one vector as claimed in claim 20.

23. A fertility-restoring maize plant, characterized in that it comprises in its genome a fertility-restoring gene linked to a “small seed” phenotype marker.

24. A maize plant homozygous for an AMS transgene and heterozygous for a fertility-restoring gene linked to a “small seed” phenotype marker, obtained from a seed as claimed in claim 3.

25. A method for the multiplication of a maize plant homozygous for an AMS transgene and heterozygous for a fertility-restoring gene linked to a “small seed” phenotype marker, comprising the steps consisting in:
a) self-fertilizing maize plants homozygous for an AMS transgene and heterozygous for a fertility-restoring gene linked to a “small seed” phenotype marker, which can be obtained by the method as claimed in claim 1,
b) selecting seeds homozygous for the AMS transgene and having a “small seed” phenotype,
c) selecting the seeds homozygous for the AMS transgene and heterozygous for a fertility-restoring gene linked to a “small seed” phenotype marker, obtained by self-fertilization of the maize plants obtained from the seeds obtained according to step b).

26. The method as claimed in claim 25, characterized in that step b) comprises densimetric separation.

27. A kit for implementing the method as claimed in claim 25, characterized in that it comprises maize seeds homozygous for an AMS transgene and heterozygous for a fertility-restoring gene linked to a “small seed” phenotype marker, and oligonucleotides specific for the AMS transgene that are useful as primers for detecting, by PCR, the seeds homozygous for an AMS transgene and heterozygous for a fertility-restoring gene linked to a “small seed” phenotype marker.

28. A maize seed homozygous for an AMS transgene and heterozygous for a fertility-restoring gene linked to a “small seed” phenotype marker, which can be obtained by the method as claimed in claim 2.

29. A method for the production of maize seeds homozygous for an AMS transgene, comprising the steps consisting in:
a) self-fertilizing maize plants derived from seeds as claimed in claim 28,
b) selecting seeds homozygous for an AMS transgene.

30. A vector, in particular a plasmid, characterized in that it contains at least one expression cassette as described in claim 15.

31. A cellular host, in particular a bacterium such as Agrobacterium tumefaciens transformed with a vector as claimed in claim 30.

32. A maize cell transformed with at least one vector as claimed in claim 30.
